(19) United States
(12) Patent Application Publication
(10) Pub. No.: US 2005/0261362 A1
(43) Pub. Date: Nov. 24, 2005

(54) Method of increasing endogenous adiponectin and leptin production

(75) Inventors: Peter J. Havel, Davis, CA (US); Kimber L. Stanhope, Davis, CA (US); Joseph L. Evans, Redwood City, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Regents of the University of California

(21) Appl. No.: 11/088,274

(22) Filed: Mar. 22, 2005

Related U.S. Application Data
(60) Provisional application No. 60/585,194, filed on Jul. 2, 2004.
(60) Provisional application No. 60/555,420, filed on Mar. 22, 2004.
(60) Provisional application No. 60/555,419, filed on Mar. 22, 2004.

(51) Int. Cl. A61K 31/4015; A61K 31/195; A61K 31/19

(52) U.S. Cl. 514/426; 514/562; 514/557

(57) ABSTRACT

A formulation for and method of enhancing adiponectin and leptin secretion is disclosed. The method comprises contacting living cells with an inhibitor of the enzyme pyruvate dehydrogenase kinase (PDHK). The PDHK inhibitor causes the cells it contacts to increase adiponectin secretion as well as increasing the production of leptin. The increased levels of adiponectin alone (or in a synergistic combination with increased leptin) provides a range of desired results including weight loss and the prevention of weight.

### CROSS-REFERENCE

This application claims the benefit of U.S. Provisional Application 60/585,194 filed Jul. 2, 2004 and the U.S. Provisional Application Nos. 60/555,420 and 60/555,419 both filed Mar. 22, 2004, all of which applications are incorporated herein by reference.

### FIELD OF THE INVENTION

The invention relates generally to the field of pharmaceuticals and methods of treatment and more particularly to pharmaceutical formulations which inhibit pyruvate dehydrodegenase kinase or elevate pyruvate dehydrogenase activity or activate malic enzyme and methods of administering such formulations in a manner which enhances adiponectin and leptin production and/or secretion from cells.

### BACKGROUND OF THE INVENTION

### Endocrine Function of Adipose Tissue

Adipose tissue produces a number of hormones involved in the regulation of energy homeostasis and substrate metabolism (Havel, Proc. Nutr. Soc., 2000, Exp. Biol. Med., 2001, Curr. Opin. Lididol., 2002, Diabetes, 2004). Adipose tissue metabolism and adipocyte hormones such as leptin and adiponectin have important roles in the regulation of fuel metabolism and energy homeostasis. Adiponectin, improves insulin sensitivity and has actions that protect the cardiovascular system from atherosclerosis. Therefore, a better understanding of the mechanisms involved in the regulation of adipocyte metabolism and the pathways controlling adiponectin and leptin production represent will lead to novel approaches for treating of obesity, the metabolic syndrome, and the consequent development of type-2 diabetes and cardiovascular disease.

### Adiponectin: Discovery and Structure

Adiponectin (also called ACRP30, adipoQ or GBP28) is a protein secreted from adipocytes. The nucleotide sequence was originally identified by four research groups using different approaches. (Scherer, P. E., et al., Journal of Biological Chemistry 270 (45): 26746-26749 (1995); Nakano, Y., et al., Journal of Biochemistry 120 (4): 803-12 (1996); Hu, E., et al. Journal of Biological Chemistry 271 (18): 10697-10703 (1996); and Maeda, K., et al., Biochemical & Biophysical Research Communications, c221 (2):286-9 (1996). The adiponectin gene is located at chromosome 3q27, a susceptibility locus for type 2 diabetes and other metabolic syndromes 12. (2002). Berg, A. H., Combs, T. P & Scherer, P. E. ACRP30/adiponectin: an adipokine regulating glucose and lipid metabolism. Trends Endocrinol. Metab. 13, 84-89 (2002). Shaprio, L. & Scherer, P. E. The crystal structure of a complement-1q family protein suggests an evolutionary link to tumor necrosis factor. Curr. Biol. 8, 335-338 (1998). Adiponectin (30 kDa) is a secreted protein expressed exclusively in differentiated adipocytes. Primary sequence analysis reveals four main domains: a cleaved amino-terminal signal sequence, a region without homology to known proteins, a collagen-like region, and a globular segment at the carboxy terminus. The globular domain forms homotrimers, and additional interactions between adiponectin collagenous segments cause the protein to form higher order structures. Adiponectin was cloned in 1995/96 and is also known as AdipoQ and Acrp30, and its human homologue has also been designated independently as apM1 and GBP28.

Adiponectin/Acrp30 protein shares sequence homology with a family of proteins showing a modular design containing a characteristic C-terminal complement factor C1q-like globular domain. The three-dimensional structure of its C-terminal globular domain is similar to that of tumor necrosis factor-α. (TNFα), even though there is no homology at the primary sequence level. T. Yokota et al (Blood, 2000; 96, 1723-1732) showed that human full-length adiponectin (produced in E. coli) specifically inhibits LPS-induced TNF-α production in human macrophages, indicating that adiponectin also may have anti-inflammatory activity. In the literature, both full-length adiponectin, (that is human adiponectin produced from E. Coli, and mouse adiponectin produced from E. Coli and mammalian cells), and globular fragments of adiponectin, (that is mouse adiponectin ACRP30 produced from E. Coli and mammalian cells), have been reported. In US patent application 20030147855 to Zolotukhin, et al. published Aug. 7, 2003 it was indicated that adiponectin cDNA was cloned into AAV serotypes 1, 2, and 5-based expression vectors. Virions containing these vectors were administered to the livers of rat subjects via portal vein injection. A single injection of 6×1011 virions of the vector caused a sustained and statistically significant reduction in body weight of the treated animals compared to the control animals. This occurred in the absence of side effects. Compared to control animals, the subject rats also exhibited reduced adipose tissue mass, reduced appetite, improved insulin sensitivity, and improved glucose tolerance. Results from a recent study suggest that circulating levels of a high molecular weight multimeric form of adiponectin composed of eighteen sub-units and its ratio to the total adiponectin is most closely linked to insulin sensitivity in animals and humans (Pajvani et al, J. Biol. Chem, 2004).

### Adiponectin Levels are Low in Obesity and Related to Cardiovascular Disease

A growing number of recent studies have been said to support the idea that adiponectin may be a hormone linking obesity with insulin resistance, type 2 diabetes, and cardiovascular disease (See Reviews, Havel, Curr. Opin. Lipidol, 2002, Havel, Diabetes, 2004). Obese subjects have low circulating levels of adiponectin (Arita, 1999). This reduction was first proposed to have a role in the pathogenesis of cardiovascular disease associated with obesity and the metabolic syndrome (Funahashi, 1999; Matsuzawa, 1999). Low adiponectin levels are associated with small LDL particle size, elevated ApoB and triglycerides (TG), and increased fasting insulin levels (Kazumi, 2002). Adiponectin also appears to have direct effects on the vascular endothelium that protect against cardiovascular disease (Okamoto, 2000; Ouchi, 2001). Genes influencing circulating adiponectin concentrations exhibit pleiotropic genetic effects on serum HDL and TG levels (Comuzzie, 2001). Further support for a cardio-protective effect of adiponectin is provided by the report that vascular injury is increased in adiponectin knockout mice (Kubota, 2002) and that adiponectin administration protects against atherosclerosis in ApoE deficient mice (Okamoto, 2002; Yamauchi, 2003a). Lastly, a large cross-sectional study demonstrated that adiponectin levels are positively related to HDL and LDL size, and negatively correlated with TG levels, independent of gender and adiposity (Cnop, 2003). Adiponectin may increase the hepatic production of HDL (Cnop, 2003). High adiponectin levels are independently associated with a reduced risk of myocardial infarction after adjusting for other risk factors (Pischon, JAMA, 2004).

### Low Adiponectin Levels are Related to Insulin Resistance/Type-2 Diabetes

Adiponectin also appears to regulate insulin action and energy homeostasis (Havel, 2002, 2004), and low levels of adiponectin have been proposed as a link between obesity and insulin resistance (Saltiel, 2001). Circulating adiponectin levels (Hotta, 2000) and adiponectin expression (Statnick, 2000) are reduced in Type 2 diabetes. Plasma adiponectin concentrations are negatively correlated with fasting insulin levels and positively correlated with insulin sensitivity (Weyer, 2001)(Cnop, 2003). Furthermore, a decline in circulating adiponectin levels coincides with the onset of insulin resistance and the development of type-2 diabetes in obese rhesus monkeys (Hotta, 2001), a model of adult-onset obesity exhibiting a progression similar to the insulin resistance syndrome observed in humans (Hansen, 1996). Genetic evidence of a role for adiponectin is provided by a genome-wide scan examining the loci influencing traits associated with obesity and insulin resistance, which identified a quantitative trait locus on chromosome 3 in the region of the adiponectin gene with LOD scores of 2.4-3.5 (Kissebah, 2000).

### Effects of Adiponectin on Insulin Sensitivity

Administration of adiponectin to mice has a number of actions, including induction of weight loss in animals on a high fat, high sucrose diet without decreasing their food intake (Fruebis, 2001) and preventing weight and fat gain in genetically obese agouti mice (Masaki, 2003). These effects were associated with reduced circulating fatty acids and increased fatty acid oxidation in muscle (Fruebis, 2001) as well as increased expression of uncoupling proteins and decreased liver triglyceride content (Masaki, 2003). Administration of recombinant adiponectin reduces hyperglycemia in mouse models of diabetes, without stimulating insulin secretion, and it enhances insulin action in isolated hepatocytes (Berg, 2001). In addition, adiponectin improves glucose tolerance in db/db mice and reduces insulin resistance associated with low adiponectin levels in mice with lipoatrophy or obesity-induced insulin resistance (Yamauchi, 2001a), although complete reversal of insulin resistance in lipoatrophic animals required co-administration of leptin (Yamauchi, 2001a). The improvements of insulin sensitivity were associated with decreased triglyceride content of muscle and liver and increased fatty acid oxidation in muscle, and were accompanied by increased expression of genes involved in fatty acid transport and utilization (Yamauchi, 2001a). Two adiponectin receptors expressed in liver and muscle were recently identified (Yamauchi, 2003b).

In addition, adiponectin receptors have been reported to expressed by pancreatic β-cells (Kharroubi et al, Biochem. Biophys. Res. Comm., 2003), suggesting that adiponectin may affect insulin secretion. The probable mechanisms of adiponectin's actions to increase systemic insulin action include a direct reduction of hepatic glucose production, and decreased liver lipid content which indirectly increases hepatic insulin sensitivity. In addition, adiponectin increases muscle glucose utilization by increasing fat oxidation and reducing circulating free fatty acid levels and muscle lipid accumulation including triglyceride stored within muscle cells (intramyocellular lipid content) (Saltiel, 2001, Havel, Diabetes 2004).

Two studies have reported insulin resistance in adiponectin knockout mice, indicating that normal adiponectin production has a role in the regulation of whole-body insulin action. In one study, there was a gene-dose effect with homozygotes being more affected than heterozygotes (Kubota, 2002), and in the other, insulin resistance was not observed unless the animals were placed on a high fat, high sugar diet (Maeda, 2002). In the latter model, insulin resistance was associated with decreased levels of fatty-acid transport protein-1 in muscle and increased TNFγ expression in adipose tissue. Virally-mediated expression of adiponectin reversed the diet-induced insulin resistance in mice (Maeda, 2002).

Lastly, circulating adiponectin concentrations are related to tyrosine phosphorylation of the insulin receptor, critical for intracellular insulin signaling, and low levels of adiponectin were predictive of a future decrease of insulin sensitivity in Pima Indians (Stefan, 2002). The insulin-sensitizing effects of adiponectin, like those of leptin, appear to involve activation of the AMP kinase pathway (Tomas, 2002; Yamauchi, 2002). Together, the available data support the idea that adiponectin increases insulin action via direct effects to lower hepatic glucose production and reduces ectopic triglyceride deposition in liver and muscle by increasing fat oxidation (Ravussin, 2002, Havel, 2004). Adiponectin's actions to increase insulin sensitivity suggest therapeutic potential for adiponectin, adiponectin secretagogues, and adiponectin receptor agonists in management of insulin resistance and type-2 diabetes (Havel, 2002, 2004). Methods designed to increase the production of both leptin and adiponectin may have synergistic or additive effects to promote tissue fat oxidation, reduce ectopic triglyceride deposition, and improve insulin sensitivity. In addition, adiponectin also has been reported to inhibit apoptosis in an insulin-secreting cell line (Rakatzi et al, Diabeteologia, 2004) suggesting that adiponectin may preserve β-cell function and insulin secretion.

### Regulation of Adiponectin Production

As discussed above, circulating adiponectin concentrations are reduced in obese mice (Hu, 1996; Yamauchi, 2001a), humans (Arita, 1999; Statnick, 2000), and rhesus monkeys (Hotta, 2001). This contrasts with the elevated plasma levels of other adipocyte derived hormones (such as leptin, TNFγ, PAI-1, and ASP) in obese subjects. Circulating adiponectin concentrations increase after weight loss in humans (Hotta, 2000; Yang, 2001). Low adiponectin levels in morbidly obese subjects are restored into the normal range after marked weight loss induced by gastric bypass surgery and related to improved insulin sensitivity and pancreatic β (Yang, 2001; Faraj, 2003; Guldstrand, 2003).

Although there is limited published information available on the mechanisms regulating adiponectin production, several studies have reported that thiazolidenediones (TZDs), agonists of PPARγ, increase adiponectin expression and circulating levels in animals (Maeda, 2001; Yamauchi, 2001b; Ye, 2003) and in humans (Hirose, 2002; Yang, 2002; Phillips, 2003). This observation suggests a mechanism by which this class of compounds acts in adipose tissue to increase whole-body insulin sensitivity (Yamauchi, 2001 b) as well as to protect against cardiovascular disease (Collins, 2001). TZDs may directly stimulate adiponectin or indirectly by increasing the number of small adipocytes producing adiponectin (Boden, 2003) or by effects on adipocyte glucose metabolism. Our experiments demonstrate that incubation of isolated adipocytes with TZDs increases adiponectin production in proportion to glucose utilization. Published reports on the effects of insulin on adiponectin are mixed. While some studies have shown that insulin increases adiponectin secretion in vitro (Bogan, 1999; Motoshima, 2002), plasma adiponectin levels do not increase during hyperinsulinemic clamps (Yu, 2002). Conflicting results have also been reported on adiponectin responses to meal ingestion (English, 2003; Peake, 2003). It is likely that insulin requires a prolonged time period to influence adiponectin production in humans. Data from Dr. Havel's laboratory demonstrated that insulin increases adiponectin secretion by isolated rat adipocytes during 96 hours in culture. The increase induced by insulin becomes significant after 48 hours in culture. Insulin increases substrate flux through pyruvate dehydrogenase (PDH) by activation of a PDH phosphatase.

As previously discussed, adiponectin is reduced in obese subjects and increased after weight loss. A plausible hypothesis to explain the reduction in obesity and the increase after weight loss is that adiponectin is preferentially produced by visceral fat as suggested by one study (Motoshima, 2002), but that large visceral adipocytes containing greater triglyceride stores produce less adiponectin. Larger adipocytes are also known to be less sensitive to the effects of insulin to stimulate glucose utilization (Foley, 1980). Data from Dr. Havel's laboratory support this hypothesis since we have found an inverse relationship between adipocyte size and adiponectin secretion from isolated adipocytes. In addition, we have demonstrated that adiponectin secretion is positively related to insulin-stimulated glucose utilization and inversely proportional to anaerobic glucose metabolism to lactate suggesting that oxidative glucose metabolism stimulates adiponectin production. Here we report that inhibition of PDH kinase to increase glucose flux through PDH stimulates adiponectin secretion. Thus, as we have previously demonstrated and reported for leptin, adipocyte glucose metabolism and flux though PDH into oxidation regulates adiponectin secretion. Thus, we propose that PDH kinase inhibitors which increase substrate flux through PDH can be identified and used to stimulate the production of endogenous adiponectin and to treat or prevent obesity, insulin resistance, dyslipidemia, type-2 diabetes, fatty liver disease (hepatic steatosis), and cardiovascular disease including atherosclerosis and coronary artery disease.

### Role of Leptin in the Regulation of Energy Homeostasis

The discovery of the adipocyte hormone, leptin, has dramatically impacted the field of obesity research. Leptin acts in the CNS to regulate food intake and energy expenditure, and in the periphery is involved in the regulation of metabolic substrate fluxes, including paracrine actions in adipose tissue itself. Normal leptin production and action are essential for maintaining energy balance. Humans and animals that cannot make leptin or respond to leptin due to receptor defects overeat and become markedly obese. Even partial leptin deficiency due to a heterozygous genetic defect in leptin production has been shown to lead to increased weight gain and adiposity (body fat content)(Farooqi et al, Nature, 2002). Circulating leptin concentrations are chronically regulated by adipose mass and acutely regulated by insulin responses to recent energy (food) intake (see Reviews, Havel, Am. J. Clin. Nutr., 1999, Proc. Nutr. Soc., 2000, Exp. Biol. Med., 2001, and Curr. Opin. Lipidol., 2002).

Increased sensations of hunger during dieting are related to decreases of circulating leptin during energy restriction (dieting) in humans (Keim et al, Am. J. Clin. Nutr., 1998) and decreased leptin production is likely to contribute to weight regain after weight loss achieved by dieting. Decreased leptin may also contribute to the fall of metabolic rate that occurs during energy-restricted diets (see Reviews, Havel, Proc. Nutr, Soc., 2000, Exp. Biol. Med., 2001). Therefore a method to stimulate endogenous leptin production (i.e. an agent that increases leptin production), in concert with dieting, could help in the induction and maintenance of weight loss by preventing leptin production and circulating leptin levels from falling.

### Regulating Leptin Production in the Treatment of Obesity and Related Metabolic Diseases

Increasing endogenous leptin production represents a novel approach to the treatment of obesity which clearly differs from the current strategy of administering exogenous leptin of recombinant origin (Heymsfield et al, JAMA. 282: 1568-1575, 1999). Since leptin has a number of actions beyond the regulation of energy balance, in addition to obesity management, a method for increasing endogenous leptin production could be useful for modulating glucose and lipid metabolism, hypothalamic-pituitary neuroendocrine function, treatment of infertility, and to promote immune function, hematopoiesis, as well as to increase angiogenesis and wound healing. For example, leptin administration was recently shown to improve glucose control and decrease serum lipids (triglycerides) in humans with diabetes due to defects in fat deposition (lipodystrophy)(Oral et al, New Engl. J. Med., 2002). A major advantage of the endogenous approach is the potential that orally-available small molecule stimulators of leptin production could be found and/or designed. Small molecule agents are considerably less costly to produce and would avoid the problems associated with the pain of daily injections and the significant injection site reactions that have been reported with subcutaneous administration of recombinant leptin (Heymsfield et al, JAMA. 282: 1568-1575, 1999). Circulating leptin levels are regulated by insulin responses to meals. Data has been generated from experiments in cultured adipocytes in vitro that indicate that glucose utilization is an important determinant of insulin-mediated leptin gene expression and leptin secretion (Mueller et al, Endocrinology, 1998). We have also shown that anaerobic metabolism of glucose to lactate does not result in increased leptin secretion (Mueller et al, Obesity Res., 8:530-539, 2000). Additional information indicates a mechanism that requires increasing the transport of substrate into the mitochondria for oxidation in the TCA cycle as a metabolic pathway by which insulin-mediated glucose metabolism regulates leptin production (Havel et al, Obesity Res., Abstract, 1999).

An important mechanism in the action of insulin to increase the flux of glucose carbon into the mitochondria for oxidative metabolism is activation of pyruvate dehydrogenase (PDH). The activity of PDH is decreased when it is phosphorylated and increases when it is dephosphorylated. Insulin increases PDH by activating a PDH phosphatase enzyme (Taylor, 1973). Another enzyme pyruvate dehydrogenase kinase (PDHK) inhibits the activity of PDH by phosphorylating the PDH enzyme complex.

### SUMMARY OF THE INVENTION

Substrate flux into oxidative metabolism increases the production of the hormone, leptin, by isolated adipocytes (fat cells). The enzyme, pyruvate dehydrogenase (PDH) which is negatively regulated by PDH kinase (PDHK) which is a key control point in the regulation of oxidative metabolism. Decreasing the activity of PDHK with biochemical inhibitors or antisense directed against PDHK increased leptin production by adipocytes. Adiponectin is another hormone produced by adipocytes that produces weight loss in some animal models, improves insulin sensitivity, reduces fat deposition in liver and skeletal muscle, protects the vascular endothelium against atherosclerosis, and increases levels of high density lipoproteins (HDL). Thus, increasing the production of endogenous adiponectin by adipose tissue aids in treating metabolic/cardiovascular disease including obesity, hepatic steatosis, insulin resistance, type-2 diabetes, dyslipidemia, and cardiovascular disease, all of which are related to the metabolic insulin resistance syndrome. Results provided here show that glucose metabolism and increased substrate flux through PDH achieved by inhibiting PDHK stimulates both adiponectin and leptin production by isolated adipocytes. Administration of inhibitors of PDHK therefore provide a method for treating obesity, insulin resistance, diabetes, and cardiovascular disease by promoting increased adiponectin production and raising circulating concentrations of adiponectin. Stimulating the production of both leptin and adiponectin with PDHK inhibitors provide synergistic effects in managing these important metabolic diseases.

A pharmaceutical formulation for and method of enhancing endogenous production and/or secretion of both adiponectin and leptin is disclosed. The method comprises administering a therapeutically effective amount of a formulation comprising a compound which inhibits pyruvate dehydrogenate kinase (PDHK) thereby contacting cells (e.g. in a living animal or human patient) with the PDHK inhibitor. The formulation of PDHK inhibitor is allowed to act on the cells for a sufficient period of time and under conditions such that endogenous adiponectin and leptin secretion by the cells is enhanced relative to the level of adiponectin and leptin secretion prior to treatment. The level of enhanced secretion may be any detectable level above the pretreatment level of the cells and/or individual being treated and not necessarily above the level of a normal cell or normal individual. Preferably the level of enhancement of each of adiponectin and leptin is 10% or more above the pretreatment level, more preferably 25% or more and still more preferably 100% more above the pretreatment level.

The biochemical and molecular (antisense) enhancements of glucose oxidation via inhibition of PDHK reported here increase leptin and adiponectin production by 20-80%. The level of enhanced adiponectin and leptin secretion can be monitored and adjustments made in dosing of the PDHK inhibitor formulation based on the measured results obtained. The adiponectin and leptin levels obtained by the treatment are each preferably therapeutic in terms of obtaining a desired overall desired result or effect not only on a cell or group of cells but on an individual, e.g. preventing weight regain after weight loss, improvement of insulin sensitivity, lowering of glucose levels in patients with type-2 diabetes, and providing protection against cardiovascular disease in at risk subjects.

Obese individuals with relatively low levels of adiponectin and/or leptin relative to normal individuals are likely to be most responsive to the treatments as provided here. Increasing the metabolic flux through pyruvate dehydrogenase (PDH) by inhibiting its regulatory enzyme PDH-kinase (PDHK) stimulates the production of adiponectin as well as the adipocyte hormone leptin. The regulatory enzyme PDH-K can be effected in different ways. For example, antisense sequences to PDH-K disrupts PDH-K production which decreases anaerobic glucose metabolism and stimulates production of either or both of adiponectin and leptin. In another example small molecules directly or indirectly inhibit the enzymatic activity of PDH-K which in turn stimulates production adiponectin and/or leptin. Both antisense and small molecule inhibitors can be used in combination to increase production of either adiponectin and/or leptin.

In addition to small molecule inhibitors of PDHK and the use of antisense it is possible to enhance endogenous production of adiponectin and/or leptin by transfecting cells with malic enzyme or by use of agents that increase the activity of malic enzyme. Any combination of two or three of these methods can be combined together to further enhance endogenous production of adiponectin and/or leptin.

In a completely different aspect of the invention there is disclosed a method of increasing endogenous production and/or secretion of both adiponectin and leptin by the administration of compounds which elevate pyruvate dehydrogenase activity. Specifically, such compounds are administered in a therapeutically effective amount over a period of three weeks or more, four weeks or more, one month or more, two months or more, six months or more, or twelve months or more. When compounds are administered over a long period of time to elevate pyruvate dehydrogenase activity over a long period of time endogenous leptin and/or adiponectin production and circulating concentrations of the two hormones are increased thereby providing a method of treating obesity, and its related diseases including insulin resistance/metabolic syndrome, hepatic steatosis, dyslipdemia, and cardiovascular disease and/or other conditions in which increasing the production and blood levels of these hormones would be beneficial.

A formulation for and method of enhancing adiponectin and leptin secretion is disclosed. The method comprises contacting living cells with an inhibitor of the enzyme pyruvate dehydrogenase kinase (PDHK). The PDHK inhibitor causes the cells it contacts to increase adiponectin secretion as well as increasing the production of leptin. The increased levels of adiponectin alone (or in a synergistic combination with increased leptin) provides a range of desired results including weight loss and the prevention of weight regain after weight loss resulting from dieting and/or exercise and/or the reduction of lipid/triglyceride deposition in liver, muscle tissue, and/or pancreatic islets resulting in increased insulin sensitivity and/or secretion. The combined increase of adiponectin, along with leptin, will prevent weight regain after weight loss and result in improvement of insulin resistance and other related metabolic diseases including type-2 diabetes, fatty liver (hepatic steatosis), and cardiovascular disease via increasing HDL production, and/or reducing inflammation and protecting the vascular endothelium against atherosclerosis.

In yet another aspect of the invention PDHK inhibitors are administered simultaneously with compounds which elevate PDH activity in order to enhance endogenous production and/or secretion of both adipnectin and leptin. The administration of both compounds is also carried out over a long period of time as described above thereby providing a method of treating obesity, enhancing weight loss and preventing weight gain after successful dieting and/or exercise, as well as the other obesity-related diseases listed above.

In this disclosure compounds which increase the activity of the enzyme pyruvate dehydrogenase (PDH) are referred to as PDH elevators.

An aspect of the invention is a formulation comprising a therapeutically effective amount of a PDHK inhibitor and a pharmaceutically acceptable carrier preferably provided in a readily administrable dosage form useful in enhancing secretion of adiponectin and/or leptin.

Another aspect of the invention is a pharmaceutical formulation (e.g., oral or injectable) comprising a carrier and a PDH-K inhibitor.

In a particular embodiment the formulation comprises an antisense sequence as the PDH-K inhibitor.

In another particular embodiment the formulation comprises an orally active small molecule PDH-K inhibitor as the active ingredient.

Another aspect of the invention is a method comprised of contacting cells with a formulation which inhibits the enzyme pyruvate dehydrogenase kinase (PDHK) in a manner which results in increasing production of adiponectin and/or leptin.

An advantage of the invention is that enhanced levels of adiponectin and/or leptin can be obtained without the administration of exogenous adiponectin and/or leptin and avoidance of the inherent problems associated with injecting exogenous proteins, including but not limited to pain at the injection site, allergic or other reactions at the injection site, and the formation of antibodies that can limit the efficacy of exogenously administered proteins.

Another advantage of the invention is that enhanced levels of adiponectin and/or leptin provide desired effects including weight loss and preventing weight gain after successful weight loss from dieting and/or exercise.

An aspect of the invention is a method for treating obesity and obesity-related metabolic diseases by stimulating endogenous production of adiponectin and/or leptin (i.e., the use of pharmacological agents that increase adiponectin and/or leptin production by adipose tissue).

Another aspect of the invention is increasing production of adiponectin and/or leptin to modulate glucose and lipid metabolism in insulin resistance/metabolic syndrome and diabetes and hyperlipidemia, to enhace hypothalamic-pituitary neuroendocrine function, to treat infertility and to promote immune function, hematopoiesis, as well as to increase angiogenesis and wound healing.

Yet another aspect of the invention is the development of new targets for compounds to accomplish the stimulation of production of adiponectin and/or leptin by increasing the metabolic flux of carbon from glucose into oxidative metabolism in the TCA cycle through a pathway involving the enzyme pyruvate dehydrogenase (PDH) by inhibiting its regulatory enzyme PDH kinase, activating PDH phosphatase, or other pathways of adipocyte metabolism such as malic enzyme and lactate dehydrogenase.

Still another aspect of the invention is the use of specific inhibitors of PDH kinase or activators of PDH phosphatase which we have demonstrated increase glucose utilization, without stimulating anaerobic glucose metabolism into lactate, and increase production of adiponectin and/or leptin from isolated cultured adipocytes.

Another aspect of the invention is the use of specific compounds to activate other metabolic pathways of adipocyte metabolism including, but not limited to, NADPH malic enzyme, lactate dehydrogenase, fatty acid oxidation, and/or cellular ATP (adenylate charge) and redox status (NADH/NAD and NADPH/NADP ratios) which are shown here to affect the regulation of production of leptin and/or diponectin by adipose tissue.

Another aspect of the invention is a method of treatment of individuals with abnormally low levels of adiponectin and/or leptin, eg. Patients with congenital, acquired, or HIV infection associated lipodystrophy.

Another aspect of the invention is a formulation manufactured for the treatment of cells and/or individuals who do not produce sufficient levels of adiponectin and/or leptin.

An aspect of the invention is a method for decreasing fat in adipocytes or the number of adipocytes comprising administering an effective amount of a pyruvate dehydrogenase-kinase (PDHK) inhibitor to adipocytes or tissue comprising adipocytes.

In another aspect of the invention the PDH-kinase inhibitor is chosen from 5,5′-Dithiobis(2-nitrobenzoate)(DTNB), Dichloroacetate (DCA), and N-ethylmaleimide (NEM).

In another aspect of the invention the PDH-kinase inhibitor is administered to a patient, may reduce appetite, increase energy expenditure, and prevent weight regain and be in an oral or parenteral formulation.

Yet another aspect of the invention is a pharmaceutical composition comprising a PDH-kinase inhibitor and a pharmaceutically acceptable carrier for administration of an effective amount of PDH-kinase inhibitor to decrease fat in adipocytes or the number of adipocytes.

Still another aspect of the invention is a method of making a formulation for decreasing fat in adipocytes or the number of adipocytes comprising adding to a pharmaceutical carrier for parenteral or oral administration an effective amount of PDH-kinase inhibitor.

Another aspect of the invention is a method of enhancing leptin production, comprising the steps of:

contacting cells with a compound which inhibits pyruvate dehydrogenase kinase; and

allowing the compound to remain in contact with the cells for a period of time and under conditions such that activity of PDHK in the cells is inhibited thereby enhancing production of adiponectin and leptin by the cells.

Yet another aspect of the invention is such a method wherein the compound is chosen from the group of examples consisting of 5,5′-Dithiobis(2-nitrobenzoate)(DTNB), Dichloroacetate (DCA), and N-ethylmaleimide (NEM).

Still another aspect of the invention is a method of enhancing production of adiponectin and leptin, comprising the steps of:

contacting cells with an exogenous nucleotide sequences encoding malic enzyme;

allowing the cells to be transfected with the nucleotide sequences in a manner such that malic enzyme is expressed and adiponectin and leptin production by the cells is enhanced.

Another aspect of the invention is a method of causing adipocytes to enhance production of adiponectin and leptin, comprising

contacting adipocytes with a pyruvate dehydrogenase kinase (PDHK) inhibitor for a period of time and under conditions such that PDHK is inhibited and production of adiponectin and leptin is enhanced.

An aspect of the invention is a formulation comprising a therapeutically effective amount of a PDH elevator and a pharmaceutically acceptable carrier preferably provided in a readily administrable dosage form useful in enhancing secretion of adiponectin and/or leptin.

Another aspect of the invention is a pharmaceutical formulation (e.g., oral or injectable) comprising a carrier and a PDH elevator.

In another particular embodiment the formulation comprises an orally active small molecule PDH-K inhibitor in combination with a PDH elevator as the active ingredient.

Another aspect of the invention is a method comprised of contacting cells with a formulation which that elevates PDH over a sufficiently long term increase production of adiponectin and/or leptin.

An advantage of the invention is that enhanced levels of adiponectin and/or leptin can be obtained without the administration of exogenous adiponectin and/or leptin, an approach which has a number of inherent limitations such as those discussed above.

Another advantage of the invention is that enhanced levels of adiponectin and/or leptin provide desired effects including weight loss and preventing weight gain after successful weight loss from dieting and/or exercise.

An aspect of the invention is a method for treating obesity by stimulating endogenous production of adiponectin and/or leptin (i.e., the use of pharmacological agents that increase leptin production by adipose tissue).

Another aspect of the invention is increasing production of adiponectin and/or leptin to modulate glucose and lipid metabolism in insulin resistance/metabolic syndrome, diabetes, hepatic steatosis, and hyperlipidemia/cardiovascular disease, to enhance hypothalamic-pituitary neuroendocrine function, to treat infertility and to promote immune function, hematopoiesis, as well as to increase angiogenesis and wound healing.

An aspect of the invention is a method for decreasing fat in adipocytes or the number of adipocytes comprising administering an effective amount of a compound which enhances pyruvate dehydrogenase activity in adipocytes or tissue comprising adipocytes.

In another aspect of the invention the PDH elevator is administered to a patient, may reduce appetite, increase energy expenditure, and to prevent weight regain after weight loss from diet and exercise and be in an oral or parenteral formulation.

Yet another aspect of the invention is a pharmaceutical composition comprising a PDH elevator and a pharmaceutically acceptable carrier for administration of an effective amount of PDH elevation to decrease fat in adipocytes or the number of adipocytes.

Still another aspect of the invention is a method of making a formulation for decreasing fat in adipocytes or the number of adipocytes comprising adding to a pharmaceutical carrier for parenteral or oral administration an effective amount of a compound which elevates PDH activity.

Another aspect of the invention is a method of enhancing leptin production, comprising the steps of:

contacting cells with a compound which elevates pyruvate dehydrogenase activity; and

allowing the compound to remain in contact with the cells for a period of time and under conditions such that activity of PDH in the cells is activated thereby enhancing production of adiponectin and leptin in the cells.

Another aspect of the invention is a method of causing adipocytes to enhance production of adiponectin and leptin, comprising

contacting adipocytes with a compound which elevates pyruviate dehydrogenase (PDH) activity inhibitor for a period of time and under conditions such that PDH activity is enhanced and production of adiponectin and leptin is enhanced.

These and other aspects, objects, advantages, and features of the invention will become apparent to those persons skilled in the art upon reading the details of the formulations and method as more fully described below.

### BRIEF DESCRIPTIONS OF THE DRAWINGS

FIG. 1 is a schematic diagram the targets, actions and the regulation of the adipocyte hormones (leptin and adiponectin).

FIG. 2 is a schematic diagram showing events involved in pyruvate dehydrogenase regulation by insulin and PDH kinase inhibitors via their effects on PDH phosphatase and PDHK, respectively.

FIGS. 3A, 3B, 3C, 3D, 3E and 3F are each bar graphs showing effects of three different PDH-kinase inhibitors to increases oxidative glucose metabolism and leptin secretion by cultured adipocytes.

FIGS. 4A and 4B are bar graphs showing the effects of antisense directed at PDH kinase to reduce anaerobic glucose metabolism and increase leptin secretion.

FIGS. 5A and 5B show graphs demonstrating that the effects of a thiazolidenedione compound (troglitazone) to increase adiponectin secretion by cultured adipocytes are related increased adipocyte glucose utilization.

FIGS. 6A, 6B, 6C and 6D show that insulin increases adiponectin secretion by cultured adipocytes and that this effects is positively related to insulin-stimulated glucose utilization and inversely related to anaerobic glucose metabolism to lactate.

FIGS. 7A, 7B and 7C show that two PDH kinase inhibitors (DTNB and DCA) or antisense directed at PDH kinase increase adiponectin secretion by cultured adipocytes.

### DETAILED DESCRIPTION OF THE INFORMATION AND DATA CONTAINED IN THE DRAWINGS

### Endocrine Function of Adipose Tissue

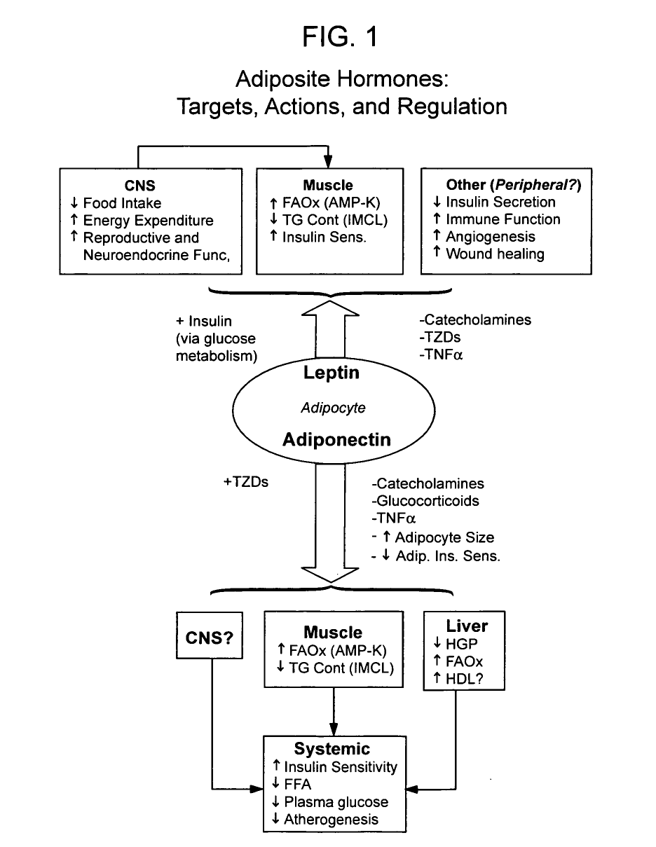

Adipose tissue produces a number of hormones involved in the regulation of energy homeostasis and substrate metabolism. Adipose tissue metabolism and adipocyte hormones such as leptin and adiponectin have important roles in the regulation of fuel metabolism and energy homeostasis (Reviews, Havel, Curr. Opin. Lipdol, 2002, Havel, Diabetes, 2004). Adiponectin, improves insulin sensitivity and has actions that would be expected to protect the cardiovascular system from atherosclerosis. Therefore, a better understanding of the mechanisms involved in the regulation of adipocyte metabolism and the pathways controlling adiponectin production represent a novel approach for the management of obesity, the metabolic syndrome and the consequent development of type-2 diabetes and cardiovascular disease (see FIG. 1).

FIG. 1—Adipocyte Hormone Overview: Leptin acts within the CNS to inhibit food intake and increase energy expenditure, perhaps via its effects to activate the sympathetic nervous system (SNS). Leptin also increases insulin sensitivity, an effect that may be largely mediated via CNS mechanisms. Leptin receptors are also found in numerous peripheral tissues where leptin exerts diverse effects. Changes of leptin secretion are primarily mediated by changes of adipocyte glucose metabolism, driven by increases and decreases of meal-induced insulin secretion. Catecholamines and thiazolidenediones (TZDs) have been reported to inhibit leptin production; however, the physiological role of these mechanisms has not been definitively established. Adiponectin improves insulin sensitivity in liver and muscle, in part by activating AMP-kinase and reduced lipid/triglyceride deposition in these insulin target tissues. Adiponectin also appears to protect the vascular endothelium against inflammation and atherosclerosis and may also raise circulating HDL levels. Adiponectin production is stimulated by thiazolidendione drugs (via PPARγ) and inhibited by catecholamines, glucocorticoids, and TNFγ. Increased adipocyte cell size (lipid content) and decreased adipocyte insulin sensitivity are associated with decreased adiponectin production. Similar to leptin production, increased oxidative glucose metabolism via pyruvate dehydrogenase increases adiponectin production by adipocytes.

### PDHK Inhibitors: Metabolic Effects and Stimulation of Leptin and Adiponectin Secretion

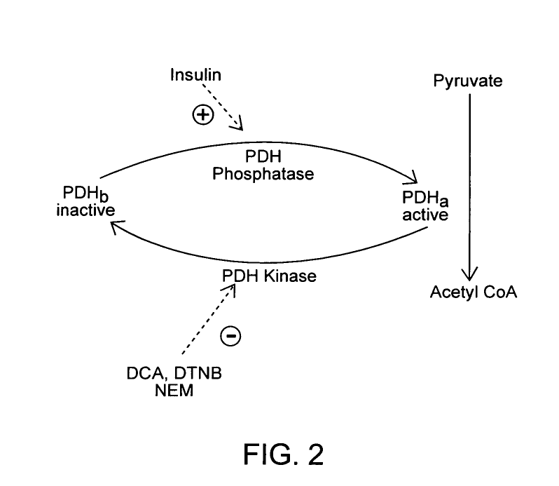

FIG. 2 is a schematic diagram of an important mechanism in the action of insulin to increase the flux of glucose carbon into the mitochondria for oxidative metabolism is activation of pyruvate dehydrogenase (PDH). The activity of PDH is decreased when it is phosphorylated and increased when it is in the dephosphorylated state. Insulin increases flux through PDH by activating PDH phosphatase. PDH kinase (PDH-K) inhibits the activity of PDH by phosphorylating the PDH enzyme complex as shown in FIG. 2.

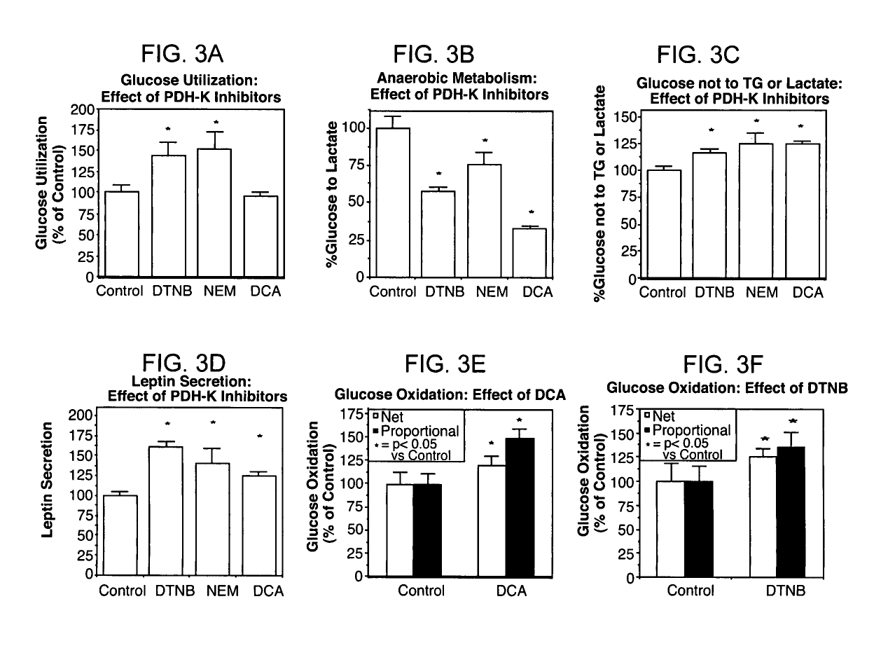

Three compounds were tested for their ability to inhibit the activity of PDH-K in an adipocyte culture system. Two PDH-K inhibitors that inactivate PDH-K by thiol-disulfide exchange (Pettit, 1982), N-ethylmaleimide (NEM 0.1 μM) (n=6) and 5,5′-Dithiobis(2-nitrobenzoate) (DTNB 100 μM) (n=11), increased adipocyte glucose utilization by 30-80% (FIG. 3A), decreased anaerobic metabolism to lactate (FIG. 3B), increased the amount of glucose not being metabolized to TG or lactate (FIG. 3C) and increased leptin production (FIG. 3D). None of the three compounds increased the proportion of glucose incorporated into TG. DCA and DTNB increased both absolute and proportional glucose oxidation as determined by incorporation of labeled glucose into CO2 (FIGS. 3E & 3F, n=6). Effects of inhibitors are represented as percent of control values (*p<0.05).

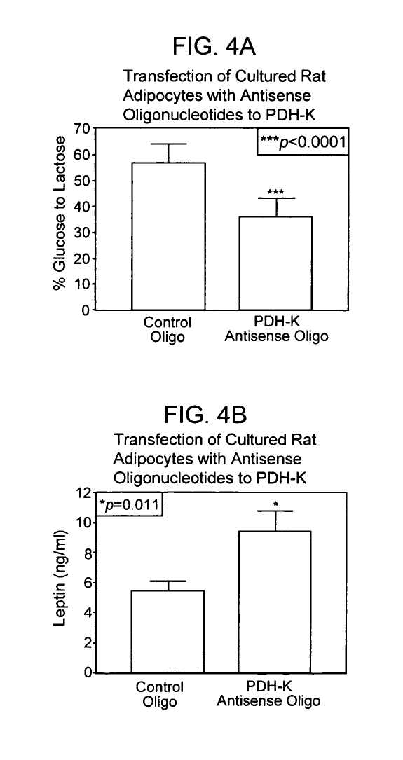

The results with biochemical inhibitors of the PDH regulatory enzyme, PDH-K, show that PDH is a critical control point in the metabolic regulation of leptin. A small molecule drug can be used to inactivate PDH-K in cultured adipocytes. The insertion of antisense oligonucleotides represents another approach (Stein, 1999; Myers, 2000). Primary adipocytes were transfected with an oligonucleotide designed to have an antisense sequence to DNA coding for PDH-K 2 and 4, or with a nonsense oligonucleotide. An adenovirus-assisted DNA transfer method was used to translocate the antisense or nonsense oligos into cultured adipocytes. In the antisense transfected cells a highly significant decrease (35%) in anaerobic glucose metabolism was observed as shown in FIG. 4A and increase (80%) in leptin secretion (FIG. 4B) (n=7). This experiment corroborates results from the biochemical studies with PDH-K inhibitors.

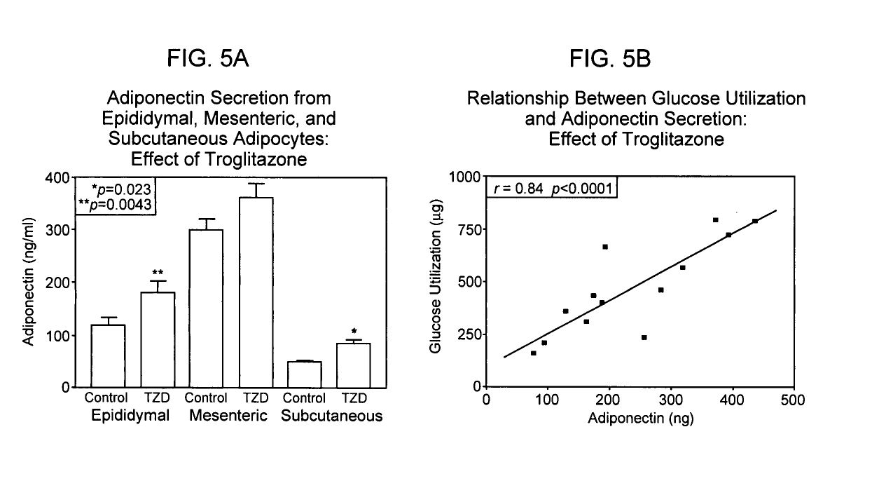

Several studies have reported that thiazolidenediones (TZDs), agonists of PPARγ, increase adiponectin expression and circulating levels in animals (Maeda, 2001; Yamauchi, 2001a; Ye, 2003). TZD (10 μM Troglitazone) stimulated of adiponectin secretion from cultured rat adipocytes from 3 different depots (n=6). Mesenteric fat produced the largest amount of adiponectin over 96 hours in culture. (FIG. 5A). Adiponectin secretion from both the control- and TZD-treated adipocytes was well correlated with the glucose utilization (control, r=0.79; p<0.001); (TZD-treated, r=0.84, p=<0.0001, FIG. 5B) showing that, similar to leptin production, glucose metabolism also is involved in the regulation of adiponectin production by adipocytes.

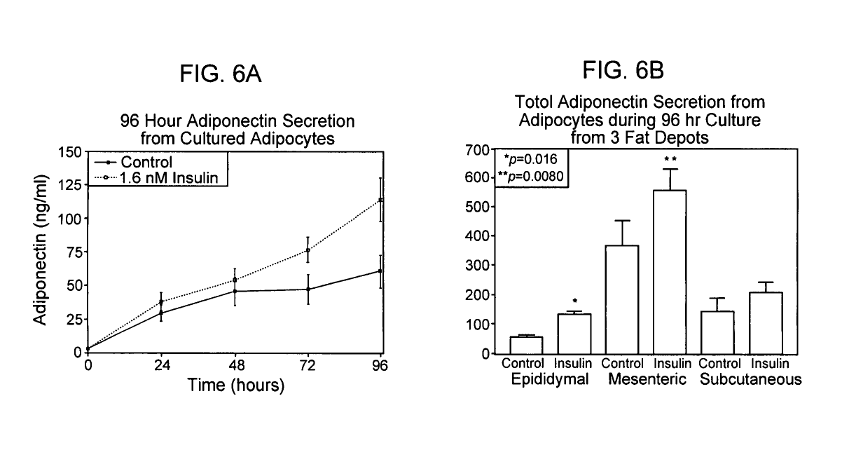

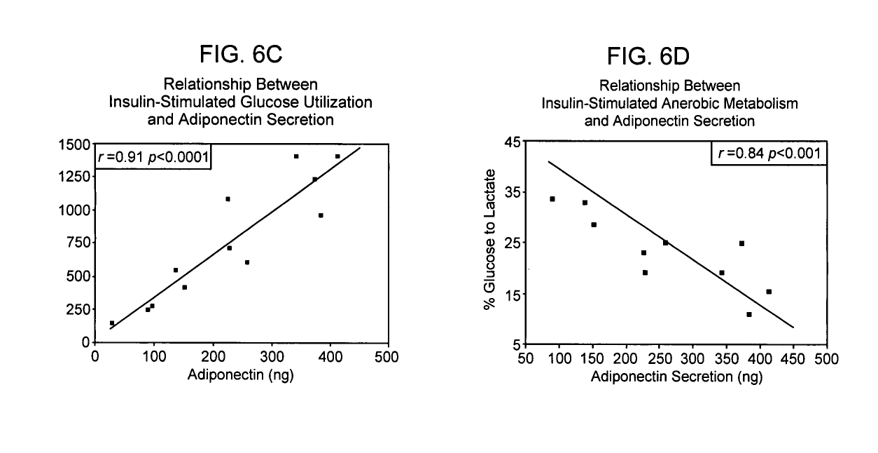

Incubation of adipocytes with 1.6 nM insulin induced a significant increase in adiponectin secretion during 96 hour culture which was significant after 48 hours (96 hr total 179.9±35.3 vs 312.3±44 ng, n=6, p=0.0005, FIG. 6A). Insulin increased adiponectin production from 3 different fat depots, however as in the experiments with troglitazone, the mesenteric depot produced the most adiponectin (FIG. 6B). Like leptin, both basal and insulin-stimulated adiponectin secretion was highly correlated to glucose utilization (r=0.91, p<0.0001) (FIG. 6C), and inversely related to the proportion of glucose metabolized to lactate (r=−0.81, p<0.0001) (FIG. 6D).

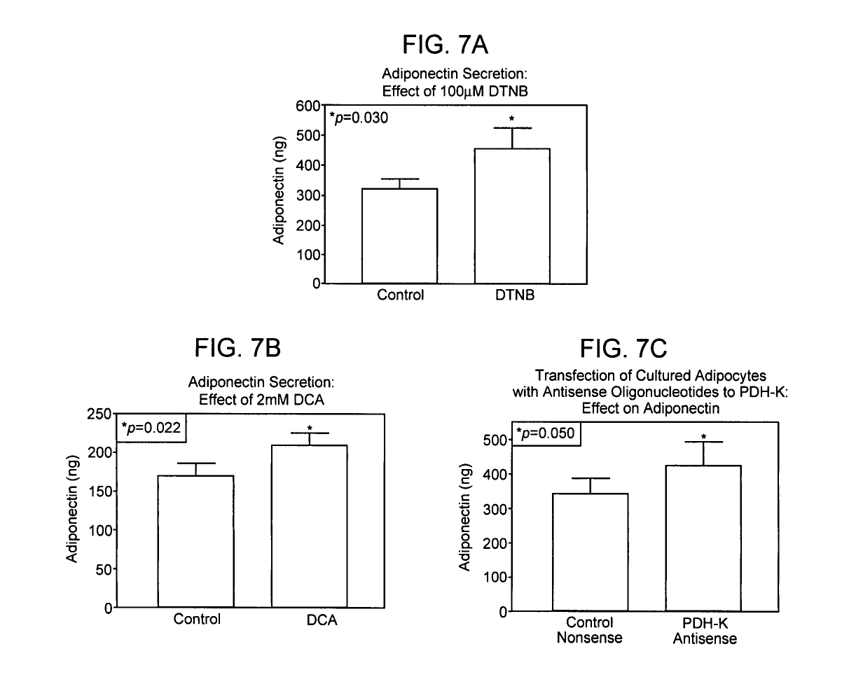

Also paralleling the regulation of leptin, adiponectin secretion by isolated cultured adipocytes was increased during 96 h culture by inhibitors of PDH-K. DTNB (100 μM) increased adiponectin secretion by 40% (p<0.03, n=6, FIG. 7A). DCA (2 mM) increased adiponectin secretion by 23% (p<0.025, n=6, FIG. 7B). The increase in adiponectin induced by DTNB was highly correlated with glucose utilization (r=0.95, p<0.004). Furthermore in adipocytes transfected with antisense directed at PDH-K, adiponectin secretion was stimulated by 24.3±8.4%, p<0.05, n=7, FIG. 7C).

The data show that there are important parallels in the regulation of the adipocyte hormones leptin and adiponectin. The production of both hormones is increased by insulin, positively linked with aerobic glucose metabolism, and inversely related to anaerobic glucose metabolism. The production of both hormones is increased by incubation of isolated adipocytes with biochemical inhibitors of PDH kinase or incorporation of antisense oligonucleotides directed to PDH kinase. PDH kinase inhibitors (FIG. 3D) and PDH kinase antisense (FIG. 4B) can be used to increase the production of leptin. The results per FIGS. 3-7 show that PDH kinase is also a promising target for increasing the production of adiponectin. These results will allow those skilled in the art to identify other potent inhibitors of PDH kinase to stimulate adiponectin production in vivo for the treatment of obesity, the metabolic (insulin resistance) syndrome, dyslipidemia, diabetes mellitus, hepatic steatosis, and cardiovascular disease.

### Summary of the Data anf its Significance

The data indicate that there are important parallels in the regulation of the adipocyte hormones leptin and adiponectin. The production of both hormones is increased by insulin, positively linked with aerobic glucose metabolism, and inversely related to anaerobic glucose metabolism. The production of both hormones is increased by incubation of isolated adipocytes with biochemical inhibitors of PDH kinase or incorporation of antisense oligonucleotides directed to PDH kinase. The use of PDH kinase inhibitors to increase the production of leptin has been addressed in a previous patent application (U.S. patent application Ser. No. 10/114,335) filed Apr. 1, 2002) and are further addressed here. These results suggest that PDH kinase is also a promising target for increasing the production of adiponectin. The next step is to identify potent inhibitors of PDH kinase to stimulate adiponectin production in vivo for the treatment of obesity, the metabolic (insulin resistance) syndrome, dyslipidemia, diabetes mellitus, hepatic steatosis, and cardiovascular disease.

### Synopsis of the Invention

A pharmaceutical formulation for and method of enhancing endogenous production and/or secretion of both adiponectin along with leptin is disclosed. The method comprises administering a therapeutically effective amount of a formulation comprising a compound which inhibits pyruvate dehydrogenate kinase (PDHK) thereby contacting cells (e.g. in a living animal) with the PDHK inhibitor. The formulation of PDHK inhibitor is allowed to act on the cells for a sufficient period of time and under conditions such that endogenous adiponectin secretion by the cells is enhanced relative to the level of adiponectin secretion prior to treatment. The level of enhanced secretion may be any detectable level above the pretreatment level of the cells and/or individual being treated and not necessarily above the level of a normal cell or normal individual. Preferably the level of enhancement of each of adiponectin and leptin is 10% or more above the pretreatment level, more preferably 25% or more and still more preferably 50 to 100% more above the pretreatment level. Preferential enhancement of the production of the bioactive, high molecular weight (18 sub-unit) form of adiponectin would be highly desirable.

The biochemical and molecular (antisense) enhancements of glucose oxidation via inhibition of PDHK reported here increase leptin production by 30-80%. The level of enhanced adiponectin and leptin secretion can be monitored and adjustments made in dosing of the PDHK inhibitor formulation based on the measured results obtained. The adiponectin and leptin levels obtained by the treatment are each preferably therapeutic in terms of obtaining a desired overall desired result or effect not only on a cell or group of cells but on an individual. The net expected result is to obtain weight loss and/or, improve insulin sensitivity, improve glucose levels in type-2 diabetes, lower the fat (triglyceride) content of the liver, raise HDL levels, and reverse or slow the progression of atherosclerotic cardiovascular disease.

Obese individuals with relatively low levels of adiponectin and/or leptin relative to normal individuals are likely to be most responsive to the treatments as provided here. Increasing the metabolic flux through pyruvate dehydrogenase (PDH) by inhibiting its regulatory enzyme PDH kinase (PDHK) stimulates the production of adiponectin as well as leptin. The regulatory enzyme PDHK can be effected in different ways. For example, antisense sequences to PDHK disrupts PDHK production which decreases anaerobic glucose metabolism and stimulates production of either or both of adiponectin and leptin. In another example small molecules directly or indirectly inhibit the enzymatic activity of PDHK which in turn stimulates production adiponectin and/or leptin. Both antisense and small molecule inhibitors can be used in combination to increase production of either adiponectin and/or leptin.

### DETAILED DESCRIPTION OF PREFERRED EMBODIMENTS

Before the present formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an enzyme” includes a plurality of such enzymes and reference to “the method” includes reference to one or more methods or steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “excipient material” and “Carrier” are used interchangeably here and intended to mean any compound forming a part of the formulation which is intended to act merely as a carrier i.e. not intended to have biological activity itself.

The terms “treating”, and “treatment” and the like are used interchangeably herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting it's development; or (c) relieving the disease, i.e. causing regression of the disease and/or it's symptoms or conditions. The invention is directed towards treating patient's suffering from all or any of obesity, metabolic syndrome, type-2 diabetes and cardiovascular disease and the related effects of any of these over long periods of time. The present invention is involved in preventing, inhibiting, or relieving adverse effects attributed to obesity, hepatic steatosis, insulin resistance/metabolic syndrome, type-2 diabetes and cardiovascular disease over long periods of time.

The terms “synergistic”, “synergistic effect” and the like are used interchangeably herein to describe improved treatment effects obtained by combining two or more compounds in formulations of the invention. Although a synergistic effect in some fields means an effect which is more than additive (e.g., one plus one equals three) in the field of treating humans to reduce obesity (or treat any disease), an additive (one plus one equals two) or less than additive (one plus one equals 1.2) effect may be synergistic. For example, if a patient is obese that patient's body fat might be reduced by a conventional orally effective compound. Further, at a different time the same patient with the same weight might be administered a different orally effective compound which compound reduced the patient's body fat. However, if both orally effective compounds are administered to the patient one would not ordinarily expect an additive effect thereby obtaining twice the fat reduction and may obtain no more of a reduction in fat than when either drug is administered by itself. If additive effects could always be obtained then obesity could be readily treated in all instances by coadministering several different types of orally effective compounds. However, such has not been found to be an effective treatment. However, in connection with the present invention coadministration of formulations of the invention comprised of a PDHK inhibitor with another active compound will provide improved results the effects which are synergistic, i.e. greater than the effects obtained by the administration of either composition by itself. Further, the PHHK inhibitor can promote production and/or release of both leptin and adiponectin, an effects that is likely to produces additive and or synergistically together.

The term “quick release formulation” refers to a conventional oral dosage formulation. Such a formulation may be a tablet, capsule or the like designed to provide for substantially immediate release of the active ingredient and includes enteric coated oral formulations which provide some initial protection to the active ingredient and thereafter allow substantially immediate release of substantially all the active ingredient. A quick release formulation is not formulated in a manner so as to obtain a gradual, slow, or controlled release of the active ingredient.

### PDHK Inhibitors Increase Leptin Production

The metabolic regulation of leptin production and the effects of PDK kinase inhibition on leptin production is first described in detail in U.S. patent application Ser. No. 10/114,335. As discussed above, the discovery of the adipocyte hormone, leptin, has dramatically impacted the field of obesity research. Leptin acts in the CNS to regulate food intake and energy expenditure, and in the periphery is involved in the regulation of metabolic substrate fluxes, including paracrine actions in adipose tissue itself. Normal leptin production and action are essential for maintaining energy balance. Humans and animals that cannot make leptin or respond to leptin due to receptor defects overeat and become markedly obese. Even partial leptin deficiency due to a heterozygous genetic defect in leptin production has been shown to lead to increased weight gain and adiposity (body fat content)(Farooqi et al, Nature, 2002). Circulating leptin concentrations are chronically regulated by adipose mass and acutely regulated by insulin responses to recent energy (food) intake (see Reviews, Havel, Am. J. Clin. Nutr., 1999, Proc. Nutr. Soc., 2000, Exp. Biol. Med., 2001, and Curr. Opin. Lipidol., 2002).

Compounds were tested for their ability to inhibit the activity of PDHK in an adipocyte culture system. Described here are results obtained with respect to affects on leptin production. Within a separate section below affects on adiponectin production are described.

At least three of these agents, 5,5′-Dithiobis(2-nitrobenzoate)(DTNB), Dichloroacetate (DCA), and N-ethylmaleimide (NEM) increase adipocyte glucose utilization, and/or decrease the anaerobic metabolism of glucose to lactate, and increase leptin production. These tests have shown that both DTNB and DCA increase glucose oxidation as assessed by the incorporation of radiolabeled glucose into CO2. In addition, molecular inactivation of PDH kinase can be carried out by transfecting cultured adipocytes with antisense targeting PDH kinase decreases anaerobic glucose utilazation and increases leptin production. Accordingly, compounds with similar mechanisms of action to inhibit PDH kinase, or that act to increase PDH phosphatase, will augment glucose metabolism in adipose tissue and increase leptin production in vivo. Such compounds are useful for treating obesity and other conditions in which increased leptin production would have beneficial effects. Since the decrease of leptin is likely to contribute to increased hunger and decreased metabolic rate during energy-restricted diets, agents that increase endogenous leptin production are useful as an adjunct to diet and/or exercise to promote weight loss and to help prevent weight regain after successful dieting.

This invention describes the concepts underlying the use of agents that promote oxidative metabolism in adipose tissue as a method for stimulating leptin production for obesity treatment. In addition, the use of inhibitors of the enzyme PDH kinase, or antisense inactivation of PDH kinase, increases substrate metabolism (e.g., oxidation) and increase leptin production. These results show that this approach (metabolic activation) can be used to increase leptin production and also show that the use of specific compounds and formulations taught here are effective in stimulating leptin production in vitro. The present inventors have also shown that other mechanisms related to metabolism in adipose tissue including, but not limited to, NADPH malic enzyme, lactate dehydrogenase, fatty acid oxidation, and/or cellular ATP (adenylate charge) and redox status (NADH/NAD and NADPH/NADP ratios) are involved in the metabolic regulation of leptin production. Knowledge of such provides targets for the development of pharmacological methods to increase leptin production. This present application extends this knowledge to the use of metabolic regulation and inhibition of PDH kinase for increasing the production of the adipocyte hormone adiponectin, and for stimulating the production of both hormones, leptin and adiponectin, together, an effect that is likely to additive or synergistic effects in the treatment of obesity and related diseases including hepatic steatosis, insulin resistance/metabolic syndrome, diabetes, and cardiovascular disease.

### Significance of the Invention: Scope and Cost of Obesity and its Related Co-Morbidities/Metabolic Syndrome

The prevalence of obesity has reached epidemic proportions in most developed countries and carries with it staggering mortality and morbidity statistics. The most recent National Health and Nutrition Examination Survey (NHANES) indicating that nearly 65% of the adult population in the United States is overweight (BMI≧25 kg/m2), and 31% is clinically obese (BMI≧30 kg/m2) (Flegal, 2002) Obesity is a well established risk factor for a number of potentially life-threatening diseases such as atherosclerosis, hypertension, diabetes, stroke, pulmonary embolism, and cancer. (Meisler J., St. Jeor S. 1996. Am J Clin Nutr. 63:409S-411S; Bray G. 1996. Endocrin Metab Clin North Amer. 25:907-919). Furthermore, it complicates numerous chronic conditions such as respiratory diseases, osteoarthritis, osteoporosis, gall bladder disease, and dyslipidemias. The enormity of this problem is best reflected in the fact that death rates escalate with increasing body weight. More than 50% of all-cause mortality is attributable to obesity-related conditions once the body mass index (BMI) exceeds 30 kg/m.sup.2, as seen in 35 million Americans. (Lee L., Paffenbarger R. 1992. JAMA. 268:2045-2049). By contributing to greater than 300,000 deaths per year, obesity ranks second only to tobacco smoking as the most common cause of potentially preventable death. (McGinnis J., Foege W. 1993. MA. 270:2207-2212).

The formula for calculating body mass index (BMI) is

$$BMI = ( \frac{weight in pounds}{( height in inches ) \times ( height in inches )} ) \times 703$$

After using the formula an indication which is below 18.5 indicates that the patient is underweight. A BMI in a range of 18.5 to 24.9 indicates that the patient has normal weight. A BMI in a range of 25 to 29.9 indicates that the patient is overweight. A BMI in the range of 30 or more indicates that the patient is obese. In accordance with the invention it is useful to treat a subpopulation of patients which are “obese” in accordance with the calculation made with the BMI formula. Particularly, it is advantageous to treat “obese” patients which are also diabetic and which may be Type I or Type II diabetics but are generally Type II diabetics who are also insulin resistant.

Even moderate obesity can contribute to the pathological characteristics of the Metabolic Syndrome (also know as Insulin Resistance Syndrome and Syndrome X), including as dyslipidemia, coronary artery disease, hypertension, insulin resistance and glucose intolerance (Grundy, 1998). The syndrome is closely associated with intra-abdominal fat deposition (i.e., central obesity or visceral adiposity) (Kissebah, 1982). It is estimated the incidence of the Syndrome has increased by more than 60% over the last decade (Bloomgarden, 2003a,b). The economic costs of obesity and its related co-morbidities of type-2 diabetes and cardiovascular complications (hyperlipidemia, hypertension, and heart disease) are enormous, close to $100 billion annually (Wolf, 1998). Treatment or prevention of obesity can help reverse or prevent type-2 diabetes and other obesity-related diseases (Doggrell, 2002). However, current medical treatment of obesity is largely ineffective, and new therapies for management of both obesity and the complications of the Metabolic Syndrome are clearly needed.

Accompanying the devastating medical consequences of this problem is the severe financial burden placed on the health care system in the United States. The estimated economic impact of obesity and its associated illnesses from medical expenses and loss of income are reported to be in excess of $68 billion/year. (Colditz G. 1992. Am J Clin Nutr. 55:503S-507S; Wolf A., Colditz G. 1996. Am J. Clin Nutr. 63:466S-469S; Wolf A., Colditz G. 1994. Pharmacoeconomics. 5:34-37). This does not include the Heater than $30 billion per year spent on weight loss foods, products, and programs. (Wolf A., Colditz G. 1994. Pharmacoeconomics. 5:34-37; Ezzati, et al. 1992. Vital health Stat [2]. 113).

In 1990, the US government responded to the crisis by establishing as a major national health goal the reduction in the prevalence cf obesity to (20% of the population by the year 2000. (Public Health Service. Healthy people 2000: national health promotion and disease prevention objective. 1990; US Department of Health and Human Services Publication PHS 90-50212). In spite of this objective, the prevalence of overweight people in the United States has steadily increased, reaching an astounding 33.0% in the most recent National Health and Nutrition Examination Survey (1988-1991). (Kuczmarski, et al. 1994. JAMA. 272:205-211). Furthermore, the mean BMI has also increased over this period by 0.9 kg/m.sup.2. This alarming trend has not occurred as the result of lack of effort. On the contrary, an estimated 25% of men, 50% of women, and 44% of adolescents are trying to lose weight at any given time. (Robinson, et al. J Amer Diabetic Assoc. 93:445-449). Rather, the 31% increase in rate and 8% increase in overweight prevalence over the past decade is a testimony of the fact that obesity is notoriously resistant to current interventions. (NIH Technology Assessment Conference Panel. 1993. Ann Intern Med. 119:764-770).

A major reason for the long-term failure of established approaches is their basis on misconceptions and a poor understanding of the mechanisms of obesity. Conventional wisdom maintained that obesity is a self-inflicted disease of gluttony. Comprehensive treatment programs, therefore, focused on behavior modifications to reduce caloric intake and increase physical activity using a myriad of systems. These methods have limited efficacy and are associated with recidivism rates exceeding 95%.

Failure of short-term approaches, together with the recent progress made in elucidating the pathophysiology of obesity, have lead to a reappraisal of pharmacotherapy as a potential long-term, adjuvant treatment. (National Task Force on Obesity. 1996. JAMA. 276:1907-1915; Ryan, D. 1996. Endo Metab Clin N Amer. 25:989-1004). The premise is that body weight is a physiologically controlled parameter similar to blood pressure, and obesity is a chronic disease similar to hypertension. The goal of long-term (perhaps life-long) medical therapy would be to facilitate both weight loss and subsequent weight maintenance in conjunction with a healthy diet and exercise. To assess this approach, the long-term efficacy of currently available drugs must be judged against that of non-pharmacological interventions alone. The latter approach yields an average weight loss of 8.5 kg at 21 weeks of treatment and only maintains 50% of the weight reduction at 4 years in 10-30% of the patients. (Wadden T. 1993. Ann Intern Med. 119:688-693; Kramer, et al. 1989. Int J Obes. 13:123-136). The few studies that have evaluated long-term (greater than 6 months) single-drug (Guy-Gran, et al. 1989. Lancet. 2:1142-1144; Goldstein, et al. 1994 Int J Obes. 18:129-135; Goldstein, et al. 1993. Obes Res. 2:92-98) or combination therapy (Weintraub M. 1992. Clin Pharmacol. Ther. 51:581-585) show modest efficacy compared with placebo in the reduction of body weight.

Fat metabolism is complicated. Multiple functions, attributed to adipose tissue include thermoregulation, energy storage, estrogen synthesis and cytokine production. While fat cells and their precursors have been the focus of many studies involving obesity, they also constitute a normal component of bone marrow. Indeed, adipocytes, hematopoiesis-supporting stromal cells, osteoblasts and myocytes appear to derive from common mesenchynmal stem cells in that tissue. Cloned preadipocyte lines with the potential for differentiation in culture have been extremely valuable for understanding the molecular regulation of differentiation. Agents that induce fat cell formation from these precursors include insulin, hydrocortisone, methylisobutylxanthine (MIBX) and ligands for peroxisome proliferator activator receptors (PPAR). On the other hand, many findings indicate that adipogenesis is also controlled through negative feedback mechanisms. For example, adipose tissue produces leptin, plasminogen activator inhibitor type 1 (PAI-1), tumor necrosis factor α (TNF-α), transforming growth factor type β (TGF-β), and prostaglmdin E2 (PGE2); agents that are thought to block fat cell formation.

Fat cells are conspicuous in normal bone marrow and have long been suspected to have an influence on hematopoiesis. Indeed, adipogenesis alters expression of extracellular matrix and cytokines in bone marrow, affecting hematopoiesis both directly and indirectlly Preadipocytes support blood cell formation in culture and fully differentiated fat cells produce less CSF-1 than their precursors. Expression of stem cell factor, interleukin-6 and leukemia inhibitory factor as well as hematopoiesis-supportive activity declined with terminal adipocyte differentiation of an embryo derived stromal line. The fat cell product, leptin, promotes osteoblast formation and hematopoiesis, while inhibiting adipogenesis.

Medications currently used to treat or prevent obesity are generally not directed at the adipocyte compartment of the tissue and generally work by either decreasing energy availability or increasing energy output. These agents can be placed into three categories based on mechanism as described below. (National Task Force on Obesity. 1996. JAMA. 276:1907-1915).

### Reduction of Energy Intake

This approach is directed at reducing food intake by decreasing appetite or increasing satiety. These ‘anorexiant’ drugs affect neurotransmitter activity by acting on either the catecholaminergic system (amphetamines, benzphetamine, phendimetrazine, phentermine, mazindol, diethylpropion, and phenylpropanolamine) or the serotonergic system (fenfluramine, dexfenfluramine, fluoxetine, sertraline, and other antidepressant selective serotonin reuptake inhibitors [SSRI]).

### Reduction in Absorption of Nutrients

Drugs in this category block the action of digestive enzymes or absorption of nutrients. An (example of this type of drug is orlistat, which inhibits gastric and pancreatic lipase activity. (Drent M., van der Veen E. 1995. Obes Res. 3 (suppl 4):623S-625S). These medications are experimental in the United States and not available for he treatment of obesity.

### Increase in Energy Expenditure

An increase in energy expenditure may be accomplished by increasing metabolic rate, for example, through changes in sympathetic nervous system tone or uncoupling of oxidative phosphorylation. Drugs that affect thermogenesis-metabolism include ephedrine alone or in combination with caffeine and/or aspirin, (Passquali R., Casimirri F. 1993 Int J Obes. 17 (suppl 1):S65-S68) and BRL 26830A, an adrenoceptor agonist. (Connacher, et al. 1992. Am J Clin Nutr. 55:258S-261 S). This class of medications is not approved by the FDA for weight control.

Currently, no single drug regimen emerges as superior in either promoting or sustaining weight loss. Surgical intervention, such as gastric partitioning procedures, jejunoileal bypass, and vagotomy, have also been developed to treat severe obesity. (Greenway F. 1996. Endo Metab Clin N Amer. 25:1005-1027). Although advantageous in the long run, the acute risk benefit ratio has reserved these invasive procedures for morbidly obese patients according to the NIH consensus conference on obesity surgery (BMI greater than 40 kg/m.sup.2). (NIH Conference. 1991. Ann Intern Med. 115:956-961). Therefore, this is not an alternative for the majority of overweight patients, unless and until they become profoundly obese and are suffering the attendant complications.

There is no medical or surgical treatment for obesity, insulin resistance/metabolic syndrome, or type-2 diabetes and cardiovascular disease that is directed at PDH-kinase.

It is therefore an object of the present invention to provide an alternative treatment to reduce obesity and the related disease of insulin resistance, glucose intolerance, the metabolic syndrome, type-2 diabetes, hepatic steatosis, dyslipidemia, and cardiovascular disease, including coronary artery disease/atherosclerosis.

Elevators of Pyruvate Dehydrogenase Activity

There are compounds which elevate pyruvate dehydrogenase activity. Some examples of those compounds are provided below.
N-(4-benzoyl-2,6-dimethylphenyl)-2-hydroxy-2-methyl-3,3,3-trifluoropropanamide,
N-(4-benzoyl-2-methylphenyl)-2-hydroxy-2-methyl-3,3,3-trifluoropropanamide,
N-(4-benzoyl-2-fluorophenyl)-2-hydroxy-2-methyl-3,3,3-trifluoropropanamide,
N-(6-chloro-3-phenylsulfonylphenyl)-2-hydroxy-2-methyl-3,3,3-trifluoropropanamide,
2-hydroxy-N-[2-methoxy-4-(4-pyridylsulfonyl)phenyl]-2-methyl-3,3,3-trifluoropropanamide,
2-hydroxy-2-methyl-N-[2-nitro-4-(phenylsulfonyl)phenyl]-3,3,3-trifluoropropanamide,
S-(−)-N-(4-benzoyl-2-methylphenyl)-2-hydroxy-2-methyl-3,3,3-trifluoropropanamide,
N-(4-benzoyl-2-chlorophenyl)-2-hydroxy-2-methyl-3,3,3-trifluoropropanamide,
N-(4-benzoyl-2-bromophenyl)-3,3-difluoro-2-(difluoromethyl)-2-hydroxypropanamide,
N-(4-benzoyl-2-bromophenyl)-2-hydroxy-2-methyl-3,3,3-trifluoropropanamide,
N-(4-benzoyl-2-cyanophenyl)-2-hydroxy-2-methyl-3,3,3-trifluoropropanamide,
N-(4-benzoyl-2-methoxyphenyl)-2-hydroxy-2-methyl-3,3,3-trifluoropropanamide,
N-(4-benzoyl-2-hydroxyphenyl)-2-hydroxy-2-methyl-3,3,3-trifluoropropanamide,
2-hydroxy-N-[2-hydroxy-4(4-pyridylsulfonyl)phenyl]-2-methyl-3,3,3-trifluoro propanamide,
N-(4-benzoyl-2,6-dimethylphenyl)-2-hydroxy-2-methyl-3,3,3-trifluoropropanamide,
N-(2-Fluoro-5-nitrophenyl)-2-hydroxy-2-trifluoromethylbutanamide,
N-(2-Fluoro-5-nitrophenyl)-2-hydroxy-2-difluoromethyl-3,3-difluoropropanamide,
3-Hydroxy-3-trifluoromethyl-1-(2-chloro-5-trifluoromethylphenyl)-4,4,4-trifluorobut-1-yne,
N-(4-benzoyl-2-methylphenyl)-2-hydroxy-2-methyl-3,3,3-trifluoropropanamide,
N-(4-benzoyl-2-fluorophenyl)-2-hydroxy-2-methyl-3,3,3-trifluoropropanamide,
2-hydroxy-N-[2-methoxy-4-(4-pyridyl-sulfonyl)phenyl]-2-methyl-3,3,3-trifluoropropanamide,
2-hydroxy-2-methyl-N-[2-nitro-4(phenyl-sulfonyl)phenyl]-3,3,3-trifluoropropanamide,
S-(−)-N-(4-benzoyl-2-methylphenyl)-2-hydroxy-2-methyl-3,3,3-trifluoropropanamide,
N-(4-benzoyl-2-chlorophenyl)-2-hydroxy-2-methyl-3,3,3-trifluoropropanamide,
N-(4-benzoyl-2-bromophenyl)-3,3-difluoro-2-(difluoromethyl)-2-hydroxypropanamide,
N-(4-benzoyl-2-bromophenyl)-2-hydroxy-2-methyl-3,3,3-trifluoropropanamide,
N-(4-benzoyl-2-cyanophenyl)-2-hydroxy-2-methyl-3,3,3-trifluoropropanamide,
N-(4-benzoyl-2-methoxyphenyl)-2-hydroxy-2-methyl-3,3,3-trifluoropropanamide,
N-(4-benzoyl-2-hydroxyphenyl)-2-hydroxy-2-methyl-3,3,3-trifluoropropanamide and
2-hydroxy-N-[2-hydroxy-4-(4-pyridylsulfonyl)phenyl]-2-methyl-3,3,3-trifluoropropanamide
N-(4-benzoyl-2-methylphenyl)-2-hydroxy-2-methyl-3,3,3-trifluoropropanamide,
N-(4-benzoyl-2-fluorophenyl)-2-hydroxy-2-methyl-3,3,3-trifluoropropanamide,
2-hydroxy-N-[2-methoxy-4-(4-pyridyl-sulfonyl)phenyl]-2-methyl-3,3,3-trifluoropropanamide,
2-hydroxy-2-methyl-N-[2-nitro-4-(phenyl-sulfonyl)phenyl]-3,3,3-trifluoropropanamide,
S-(−)-N-(4-benzoyl-2-methylphenyl)-2-hydroxy-2-methyl-3,3,3-trifluoropropanamide,
N-(4-benzoyl-2-chlorophenyl)-2-hydroxy-2-methyl-3,3,3-trifluoropropanamide,
N-(4-benzoyl-2-bromophenyl)-3,3-difluoro-2-(difluoromethyl)-2-hydroxypropanamide,
N-(4-benzoyl-2-bromophenyl)-2-hydroxy-2-methyl-3,3,3-trifluoropropanamide,
N-(4-benzoyl-2-cyanophenyl)-2-hydroxy-2-methyl-3,3,3-trifluoropropanamide,
N-(4-benzoyl-2-methoxyphenyl)-2-hydroxy-2-methyl-3,3,3-trifluoropropanamide,
N-(4-benzoyl-2-hydroxyphenyl)-2-hydroxy-2-methyl-3,3,3-trifluoropropanamide and
2-hydroxy-N-[2-hydroxy-4(4-pyridylsulfonyl)phenyl]-2-methyl-3,3,3-trifluoropropanamide,
N-(4-benzoyl-2-methylphenyl)-2-hydroxy-2-methyl-3,3,3-trifluoropropanamide,
N-(4-benzoyll-2-fluorophenyl)-2-hydroxy-2-methyl-3,3,3-trifluoropropanamide,
2-hydroxy-N-[2-methoxy-4-(4-pyridyl-sulfonyl)phenyl]-2-methyl-3,3,3-trifluoropropanamide,
2-hydroxy-2-methyl-N-[2-nitro-4-(phenyl-sulfonyl)phenyl]-3,3,3-trifluoropropanamide,
S-(−)-N-(4-benzoyl-2-methylphenyl)-2-hydroxy-2-methyl-3,3,3-trifluoropropanamide,
N-(4-benzoyl-2-chlorophenyl)-2-hydroxy-2-methyl-3,3,3-trifluoropropanamide,
N-(4-benzoyl-2-bromophenyl)-2-hydroxy-2-methyl-3,3,3-trifluoropropanamide,
N-(4-benzoyl-2-cyanophenyl)-2-hydroxy-2-methyl-3,3,3-trifluoropropanamide,
N-(4-benzoyl-2-methoxyphenyl)-2-hydroxy-2-methyl-3,3,3-trifluoropropanamide,
N-(4-benzoyl-2-hydroxyphenyl)-2-hydroxy-2-methyl-3,3,3-trifluoropropanamide and
2-hydroxy-N-[2-hydroxy-4-(4-pyridylsulfonyl)phenyl]-2-methyl-3,3,3-trifluoropropanamide,
N-(4-benzoyl-2-fluorophenyl)-2-hydroxy-2-methyl-3,3,3-trifluoropropanamide,
2-hydroxy-N-[2-methoxy-4-(4-pyridyl-sulfonyl)phenyl]-2-methyl-3,3,3-trifluoropropanamide,
2-hydroxy-2-methyl-N-[2-nitro-4-(phenyl-sulfonyl)phenyl]-3,3,3-trifluoropropanamide,
N-(4-benzoyl-2-chlorophenyl)-2-hydroxy-2-methyl-3,3,3-trifluoropropanamide,
N-(4-benzoyl-2-bromophenyl)-3,3-difluoro-2-(difluoromethyl)-2-hydroxypropanamide,
-(4-benzoyl-2-bromophenyl)-2-hydroxy-2-methyl-3,3,3-trifluoropropanamide,
N-(4-benzoyl-2-methoxyphenyl)-2-hydroxy-2-methyl-3,3,3-trifluoropropanamide,
N-benzoyl-2-hydroxyphenyl)-2-hydroxy-2-methyl-3,3,3-trifluoropropanamide and
2-hydroxy-N-[2-hydroxy-4-(4-pyridylsulfonyl)phenyl]-2-methyl-3,3,3-trifluoropropanamide,
   and pharmaceutically acceptable in vivo cleavable esters of said compounds, and pharmaceutically acceptable salts of said compounds and said esters.

There are other compounds known in the art which elevate pyruvate dehydrogenase activity such as those disclosed within U.S. Pat. No. 6,369,273 issued Apr. 9, 2002; U.S. Pat. No. 6,498,275 issued Dec. 24, 2002 and U.S. Pat. No. 6,552,225 issued Apr. 22, 2003 all of which are incorporated herein by reference in their entirety to disclose pyruvate dehydrogenase activity as well as formulations containing such. It is also pointed out that these patents cite numerous publications and other patents which also disclose and describe compounds which elevate pyruvate dehydrogenase activity. These additional patents and publications are also incorporated herein by reference in their entirety.

In accordance with applicants' invention such compounds which elevate pyruvate dehydrogenase activity can be administered in a pharmaceutically acceptable formulation comprised of a carrier and the active compound and administered over a long period of time while monitoring the patient in order to determine the effects of the compound on enhancing endogenous production and/or secretion of both adiponectin and leptin. In accordance with applicants' invention it is desirable to administer a therapeutically effective amount of such a PDH elevator on a daily basis of once a day, twice a day, three times a day or four times a day dosing over a period of three weeks or more, four weeks or more, one month or more, two months or more, six months or more or twelve months or more to obtain the desired effect with respect to treating obesity, managing weight gain or other desired effects as described herein.

Scope, Costs, and Complications of Obesity:

Obesity is a serious, costly, and growing medical problem in the United States and throughout much of the world. Using the most stringent criteria, more than half of U.S. men and women age 20 and older are considered overweight (a body mass index (BMI)≧25 kg/m²), and nearly one-fourth are clinically obese (BMI≧30 kg/m²) (Wickelgren, 1998, Hill, 1998). The economic costs of obesity and its related co-morbidities of Type-2 diabetes and cardiovascular complications (hyperlipidemia, hypertension, and heart disease) are enormous; close to $100 billion (Wolf, 1998).

Since the prevalence of obesity is increasing, obesity-related diseases will demand a growing portion of the nation's health-care resources in the next century unless this troublesome trend can be reversed. Treatment or prevention of obesity is likely to reverse or prevent the onset of Type-2 diabetes and other obesity-related diseases. However, since current medical treatments of obesity are largely ineffective, new approaches to obesity management are clearly needed.

Although significant weight loss can often be achieved through the implementation of energy-restricted diets and/or exercise, the success rate in maintain the weight loss is very low. Therefore, new therapies for obesity management are clearly needed. Adipose tissue metabolism and the adipocyte hormone, leptin, have a central role in the regulation of fuel metabolism and energy balance. Accordingly, a better understanding of the mechanisms involved in the regulation of adipocyte metabolism and leptin production may lead to new approaches for controlling obesity. The present invention provides pharmacological agents which augment leptin production and prevent the decrease of leptin during dieting and therefore attenuate the increase of appetite (hunger) and decline in energy expenditure (i.e., activity and metabolic rate) associated with restriction of energy intake.

Leptin: Importance in Human Energy Balance:

The adipocyte hormone leptin (Zhang et al, 1995) is involved in the regulation of body weight via its central actions on energy intake and expenditure (Caro et al, 1996). Evidence of a role for leptin as a hormonal signal from peripheral adipose stores to the central nervous system has primarily been based on rodent studies. However, more recent evidence, including reports that leptin deficiency (Montague et al, 1997), or defects in the leptin receptor (Clement et al, 1998), cause increased appetite leading to overeating and extreme obesity in humans demonstrates that leptin is a critical regulator of energy balance in humans as well as rodents (See Review, Havel, Am. J. Clin. Nutr., 1998, Am. J. Clin. Nutr., 1999, Proc. Nutr. Soc., 2000, Exp. Bio. Med. 2001, Curr. Opin. Lipidol., 2002).

Leptin Responses to Energy Restriction:

Circulating leptin concentrations decrease during energy restriction in humans and the decrease is much larger than would be expected for the smaller changes in body fat content (Dubuc, 1998). A decrease of leptin is linked to increased appetite during an energy-restricted diet in human subjects (Keim, 1998). Hyperphagia, in insulin-deficient diabetic rats is mediated by a decrease of circulating leptin (Sindelar, 1999). Furthermore, the fall of resting energy expenditure in response to fasting in rodents is prevented by leptin administration (Doring, 1998). Thus, decreased leptin production increases appetite and food drive, and contributes to the lowering of metabolic rate that is observed in humans during an energy-restricted diet. Since weight maintenance at a lower level of body adiposity is more difficult than achieving initial weight loss, a treatment which prevents the fall in leptin that accompanies energy-restricted diets will be beneficial in sustaining weight loss after successful dieting. Increasing leptin production during the period of dynamic weight loss will also increase the effectiveness of diet/exercise regimens in initiating weight loss.

Peripheral Actions of Leptin:

Leptin has a number of effects other than its central actions to reduce food intake and increase energy expenditure. There are leptin receptors in many peripheral tissues (see review, Tartaglia, 1997), including the liver, kidney, adipose tissue, ovary, and gastrointestinal tract. Leptin appears to have peripheral actions on fuel metabolism and substrate flux (Rossetti et al, 1997, Barzilai et al, 1997). That these actions may have profound long-term effects is suggested by studies showing that two weeks of hyperleptinemia after leptin gene transfection (Chen et al, 1997) or during leptin infusion from osmotic minipumps (Barzilai et al, 1997) led to a marked loss of body fat in rats, whereas pair-fed animals exhibited much more modest reductions of body fat.

Other Important Biological Actions of Leptin:

Leptin is also involved in regulating reproductive function (see Review, Cunningham et al, 1999) since ob/ob mice lacking leptin are infertile, but fertility is restored by leptin treatment (Chehab et al, 1996). Obese human patients with leptin deficiency exhibit hypogonadism (Strobel et al, 1998). Furthermore, leptin administration has been shown to accelerate the onset of puberty in rodents (Barash et al, 1996, Cheung et al, 1997, Chehab et al, Science, 1997). It has been proposed that leptin acts as a general signal of low energy status to the neuroendocrine axes; leptin administration reverses the changes of thyrotropin, ACTH, and gonadotropins in response to fasting in mice (Ahima et al, 1996) and energy-restricted rats (Kras et al, 2000).

Humans with leptin receptor defects are not only obese, but have impaired growth hormone and thyrotropin secretion (Clement et al, 1998). Low leptin levels, resulting from very low amounts body fat and decreased food intake, contribute to amenorrhea in women athletes (Laughlin et al, 1997) or anorexic patients (Kopp et al, 1997). Leptin has additional centrally- and peripherally-mediated effects on carbohydrate and lipid metabolism. Leptin administration has been shown to decrease glucose and hemoglobin A1c levels, and reduce plasma triglycerides in humans with low leptin levels, hyperlipidemia, and insulin-resistant diabetes resulting from lipodystophy (Oral, New Engl. J., Med., 2002). Therefore, increasing endogenous leptin production would be useful in the treatment of some forms of hyperlipidemia and diabetes. Other potential functions of leptin include direct inhibitory effects on insulin secretion (Kieffer et al, 1997, Emilsson et al, 1997, Ahren & Havel, 1999), actions on adrenal function (Bornstein et al, 1997, Cao et al, 1997), angiogenesis (Bouloumie et al, 1998, Sierra-Honigmann et al, 1998), hematopoiesis (Gainsford et al, 1996), pulmonary function (O'donnell et al, 1999) and immune function (Lofreda et al, 1998, Lord et al, 1998). Therefore, a pharmacological method which increase leptins production will provide therapeutic value in treating a number of conditions such as infertility or impaired function of the hypothalamic-pituitary neuroendocrine axes, including gonandotrophic, thyrotrophic and adrenocorticotrophic function.

In addition, to its potential utility in weight loss and weight loss maintenance in obesity, increasing endogenous leptin production through modulation of adipocyte metabolism provides a useful treatment for promoting immune function, angiogenesis and wound healing, and hematopoiesis.

Regulation of Leptin Production in Vivo:

Circulating leptin concentrations are correlated with adiposity in humans and animals (Maffei, et al, 1995, Ahren et al, 1997, Havel et al, 1996). However, adiposity is not the sole determinant of circulating leptin concentrations since plasma leptin decreases after fasting (Ahren et al, 1997, Weigle et al, 1997) and increases after refeeding (Weigle et al, 1997) with only minor changes of body adiposity. In humans, a diurnal pattern of leptin secretion has been described with the highest concentrations occurring between midnight and 2:00 A.M (Sinha et al, 1996). This nocturnal peak is related to insulin responses to meals (Laughlin and Yen, 1997, Saad et al, 1998), is entrained by meal timing (Schoeller et al, 1997), and does not occur if the subjects are fasted (Boden et al, 1996).

A weight-maintaining low fat/high carbohydrate diet increases energy expenditure in women (Havel et al, 1996). Furthermore, feeding a low fat/high carbohydrate diet results in significant weight loss, even when it is consumed ad libitum (Havel et al, 1996). Increases of circulating leptin and insulin in response to high carbohydrate feeding appear to lower the regulated level of adiposity by producing small but prolonged increases of metabolic rate, an effect that is likely to be mediated by increases of insulin and leptin.

Meals high in carbohydrate content result in higher leptin concentrations over a 24 hr period than high fat meals. This is shown in a study measuring leptin over 24 h in 19 normal weight women consuming either high fat/low carbohydrate meals (60%/20%) or low fat/high carbohydrate (20%/60%) meals (Havel et al, 1999). Meal-associated plasma insulin and glucose excursions were larger after low fat/high carbohydrate meals. Plasma leptin concentrations were higher 4-6 hr after breakfast and lunch and the nocturnal rise was augmented after low fat/high carbohydrate meals compared with high fat/low carbohydrate meals. Adipocyte glucose metabolism regulates leptin expression and secretion, increases of dietary fat content reduce leptin production via a mechanism that is likely to be related to decreased insulin-mediated glucose metabolism in adipose tissue. This reduction of leptin levels contributes to the effects of high fat diets to promote increased energy intake, weight gain, and obesity in animals (Ahren et al, 1997, Hill et al, 1992, Surwit et al, 1997) and humans (Horton et al, 1995, Tataranni et al, 1997) and the effect of low fat/high carbohydrate diets to promote weight loss (Havel et al, 1996). The effects of dietary carbohydrate to stimulate leptin production can be augmented by administering a pharmacological agent acting at the level of the adipocyte. Thus, administration of such an agent makes it possible to promote and maintain weight loss induced by low fat or energy-restricted diets by lowering the regulated level of body adiposity.

Evidence for a Role of Insulin-Mediated Glucose Metabolism in Regulating Leptin Production:

Glucose is an important regulator of leptin expression and secretion. This is demonstrated by showing that increases of leptin (ob) mRNA after glucose administration in mice are well correlated with plasma glucose concentrations (Mizuno et al, 1996). Such is further demonstrated by showing that the infusion of small amounts of glucose to prevent the decline of glycemia during fasting in humans also prevents the decrease of plasma leptin (Boden et al, 1996). Further the decrease of plasma leptin during marked caloric restriction in humans is better correlated with the decrease of plasma glucose than with changes of insulinemia (Dubuc et al, 1998). Still further low plasma leptin levels are acutely increased by insulin administration in proportion to the degree of glucose lowering in insulin-deficient diabetic rats (Havel et al, 1998) or in insulin-dependent diabetic human subjects (Havel et al, 1997). Lastly, high carbohydrate meals, which produce larger insulin and glycemic excursions, increase 24 hr plasma leptin concentrations in human subjects when compared with equicaloric high fat meals (Havel et al, 1999). Thus, insulin is a physiological regulator of leptin production. However, in experiments with insulin infusion, it is necessary to infuse significant amounts of glucose along with insulin to prevent hypoglycemia. Therefore, it was previously unclear whether the increased leptin production during insulin and glucose administration is due to a direct effect of insulin per se, or might be mediated indirectly via insulin's actions to increase glucose uptake and metabolism in adipose tissue.

Effects of Insulin-Mediated Glucose Transport and Glycolysis:

A number of early in vitro studies conducted in isolated adipocytes found that insulin stimulated leptin expression and secretion (Mueller, 1998; Russell, 1998). The present invention shows that glucose metabolism has a role in mediating insulin-induced leptin expression and secretion as opposed to a direct role on the insulin signal transduction pathway. Among the numerous actions of insulin to stimulate glucose utilization are the effects of insulin to stimulate glucose transport into cells by increasing the translocation of glucose transporters (GLUT4) to the plasma membrane. In addition insulin increases the flux of glucose through glycolysis primarily at the level phosphofructokinase (PFK) by increasing enzymatic production of fructose-2,6 bisphosphate, an allosteric activator of PFK (Tepperman, 1980)(Schematic Diagram 1).

Adipocyte Culture System:

To investigate the role of adipocyte metabolism in regulating leptin production, the present invention provides a culture system in which freshly isolated mature rat adipocytes are maintained in culture anchored to a basement membrane matrix (Matrigel) or collagen. Although all in vitro systems have inherent advantages and disadvantages, advantages of this system compared with cultures containing free-floating adipocytes are (1) that the matrix simulates their normal basement membrane attachment and (2) that the cells are maintained in close proximity to each other, allowing direct cell-to-cell contact. Together the cell contact and basement membrane attachment help to maintain differentiation, since adipocytes have a strong tendency to dedifferentiate in long-term (>24 h) culture. Furthermore, the adipocytes in this system are not exposed to toxic levels of oxygen at the interface of the media and the incubator atmosphere, as opposed to free-floating adipocytes, which aggregate at the surface of the media. An advantage of the system over those containing minced adipose tissue is that all of the cells in the culture are equally exposed to the nutrients and the oxygen dissolved in the media. Thus, although clearly different from the in vivo situation, we believe that this system provides a more physiological environment than most systems for maintaining adipocytes in long-term culture.

The present invention demonstrates that glucose utilization by adipocytes is required for insulin-stimulated leptin expression and secretion. The results obtained show that leptin secretion is proportional to the rate of glucose utilization. Other experiments demonstrate that leptin secretion and ob gene expression are suppressed when glucose transport and phosphorylation are inhibited with 2-deoxy-D-glucose (2-DG) treatment. Furthermore, leptin expression and secretion are reduced when glycolysis was suppressed with sodium fluoride (Mueller, 1998). Other inhibitors of glucose transport and utilization had similar effects to inhibit leptin production. The suppression of leptin production by all of the agents examined was proportional to actions of the compounds to inhibit adipocyte glucose utilization.

FIGS. 6A-6D illustrate the effects of increasing concentrations of insulin within a physiological range (0.16-1.6 nM) on adipocyte metabolism and leptin production. As outlined above, insulin induces a concentration dependent increase in leptin secretion (FIG. 6A) and glucose utilization (FIG. 6B).

Insulin (0.1 to 10 nM) also increases the transcriptional activity of a luciferase construct driven by the leptin promoter when it is transfected into 3T3-L1 adipocytes, an effect that is completely blocked when glucose metabolism is inhibited with 2-DG (FIG. 12A). In contrast the activity of a control plasmid expressing β-galactosidase was unaffected by insulin or 2-DG (FIG. 12B), suggesting that insulin and 2-DG are not exerting generalized effects on cellular transcriptional activity (See Moreno-Aliaga et al, Biochem. Biophy. Res. Comm., 2001). These results from experiments in 3T3-L1 cells have now been replicated in primary adipocytes in which the activity of a transfected construct of the leptin promoter is increased by insulin and the effect of insulin is blocked by inhibiting glucose metabolism with 2-DG. In contrast the activity of the a control plasmid was unaffected by insulin or 2-DG.

Role of Aerobic Metabolism:

Insulin: 1) decreases the proportion of glucose that is anaerobically metabolized to lactate (FIG. 6C), 2) does not alter the proportion of glucose that is incorporated into triglyceride (FIG. 6D), and 3) by subtraction increases the proportion of glucose that is not converted to lactate or triglyceride (FIG. 6E).

This glucose was subjected to mitochondrial oxidation and the present invention shows that insulin at a concentration of 1.6 nM markedly increases glucose oxidation as assessed by the incorporation of ¹⁴C-labeled glucose into CO₂ (FIG. 6F). The Examples show that glucose transport per se is not the regulatory step in leptin production by adipocytes. Rather, glucose transport and phosphorylation are necessary in order for glucose to be further metabolized. The Examples also show that leptin secretion is inversely related to the rate of conversion of glucose to lactate (FIG. 7A). This shows that anaerobic metabolism of glucose does not stimulate leptin production. Additional studies with metformin revealed that metformin inhibits leptin secretion by diverting glucose into an anaerobic pathway generating lactate (FIGS. 7B-D). Based on these results and other factors we have deduced that the metabolism of glucose beyond pyruvate, to a fate other than lactate, causes effects on glucose metabolism to stimulate leptin production. Further other pathways of glucose metabolism which are stimulated by insulin in adipose tissue particularly mitochondrial metabolism are involved in the regulation of leptin production by glucose.

Role of Glucose Oxidation:

Other data shows the connection between oxidative metabolism and the regulation of leptin secretion. The uncoupling agent, dinitrophenol (DNP), at low concentrations, markedly increases glucose utilization (FIG. 9A), glucose and fatty acid oxidation (FIGS. 9B and 9C), and stimulates leptin secretion (FIG. 9D). The increase in glucose utilization is a compensatory response to reduced ability to generate ATP. Thus, under these conditions, the flux of substrate into and through the TCA cycle is increased. Another method for increasing the flux of carbon from glucose into the TCA cycle is to increase the activity of pyruvate dehydrogenase (PDH)(see FIG. 3 for an overview of PDH regulation).

The enzyme PDH kinase (PDHK) is a negative regulator of PDH activity. When PDH is phosphorylated by PDHK, its activity is decreased and less glucose carbon can enter the mitochondria for oxidation through the TCA cycle. Insulin increases PDH activity by activating a PDH phosphatase enzyme (Taylor, 1973), which dephosphorylates PDH (see FIG. 3). Therefore if PDHK is inhibited or if PDH phosphatase is activated, PDH activity will increase and more glucose can be oxidized. This would stimulate leptin production.

Role of PDH and PDH-K:

The results provided here show that specific inhibitors of PDHK increase PDH activity in adipocytes and stimulate leptin production. PDHK inhibitors are described in Proc. Natl. Acad. Sci. USA, 19:3945-3948 (1982). The inhibitors, N-ethylmaleimide (NEM) and 5,5′-Dithiobis(2-nitrobenzoate)(DTNB) were tested in the adipocyte culture system, FCC 136, Vol. 3, page 127). The analyses of the glucose and lactate results from that experiment showed that DTNB at a concentration of 100 μM and NEM at a concentration of 0.1 μM increased insulin-mediated glucose utilization (FIG. 8A) without any increase of lactate production. Specifically these two PDHK inhibitors decreased the proportion of glucose carbon released as lactate (FIG. 8B), without affecting the proportional incorporation of labeled glucose into triglyceride. By subtraction it was shown that these inhibitors increased the proportional glucose flux into oxidation (FIG. 8C).

The same concentrations of DTNB (100 μM) increased leptin production by approximately 60% and NEM (0.1 μM) increased leptin secretion by 25% (FIG. 8D).

Another compound reported to be a PDH kinase inhibitor, dichloroacetate (DCA), at a concentration of 2 mM, did not increase total glucose utilization (FIG. 8A), but markedly lowered anaerobic metabolism of glucose to lactate (FIG. 8B), did not increase the proportional incorporation of labeled glucose into triglycerides, and by subtraction increased glucose oxidation (FIG. 8C), and increased leptin secretion by 20-25% (FIG. 8D).

These results provide strong evidence that increasing glucose carbon flux into the mitochondria through PDH for oxidative metabolism by inhibiting PDHK is a viable approach for increasing leptin production by adipose tissue. The effects of DTNB, NEM, and DCA on glucose utilization, lactate production, the proportional anaerobic conversion of glucose to lactate, and leptin secretion are summarized in Tables 1-3, respectively. Therefore, PDHK inhibitors enhance leptin production and are useful in the management, including treatment of obesity and the prevention of weight regain after weight loss.

TABLE 1
Effects of 5,5′-Dithiobis(2-nitrobenzoate)(DTNB) in the presence of 0.48 nM insulin
on glucose utilization, lactate production, the percentage of glucose carbon taken up that was
released as lactate, and leptin production by isolated rat adipocytes over 96 hours in culture.

| DTNB (μM) + | | Lactate | | Leptin (ng) | % ΔLeptin | [Leptin] | % Δ[Leptin] |
|---|---|---|---|---|---|---|---|
| Insulin (0.48 | Glucose Uptake | Produced (μg) | Glucose to | Produced | Produced | (ng/ml) | (ng/ml) |
| nM) | (μg) over 96 h | over 96 h | Lactate (%) | over 96 h | over 96 h | at 96 h | at 96 h |
| Control | 1035 ± 86 | 231 ± 27 | 22.6 ± 1.8 | 37.1 ± 2.2 | — | 10.9 ± 0.6 | — |
| (n = 11) | | | | | | | |
| 100.0 (n = 11) | 1384 ± 94 | 179 ± 22 | 13.1 ± 1.4 | 55.6 ± 2.9 | — | 17.2 ± 0.9 | — |
| Change in | +348 ± 114 | −53 ± 25 | −9.5 ± 1.0 | +18.4 ± 2.3 | +57.3 ± 5.8 | +6.3 ± 0.8 | +60.2 ± 8.3 |
| Parameter | | | | | | | |
| t-value | 3.05 | 2.12 | 9.50 | 8.00 | 9.88 | 7.88 | 7.25 |
| p-Value | p < 0.01 | 0.05 | P < 0.0001 | P < 0.0001 | P < 0.0001 | P < 0.0001 | P < 0.0001 |

TABLE 2
Effects of N-ethylmaleimide (NEM) in the presence of 0.48 nM insulin on glucose utilization,
lactate production, the percentage of glucose carbon taken up that was released as lactate,
and leptin production by isolated rat adipocytes over 96 hours in culture.

| NEM (μM) + | | Lactate | | Leptin (ng) | % ΔLeptin | [Leptin] | % Δ[Leptin] |
|---|---|---|---|---|---|---|---|
| Insulin (0.48 | Glucose Uptake | Produced (μg) | Glucose to | Produced | Produced | (ng/ml) | (ng/ml) |
| nM) | (μg) over 96 h | over 96 h | Lactate (%) | over 96 h | over 96 h | at 96 h | at 96 h |
| Control (n = 6) | 970 ± 80 | 188 ± 15 | 20.6 ± 2.0 | 49.2 ± 4.5 | — | 14.5 ± 1.3 | — |
| 0.1 (n = 6) | 1399 ± 132 | 178 ± 11 | 13.6 ± 1.5 | 63.4 ± 8.2 | — | 18.4 ± 2.4 | — |
| Change in | +348 ± 114 | −10 ± 13 | −6.9 ± 2.3 | +14.2 ± 4.2 | +26.6 ± 6.2 | +4.0 ± 1.0 | +25.3 ± 6.3 |
| Parameter | | | | | | | |
| t-value | 3.05 | 0.77 | 3.0 | 3.38 | 4.29 | 4.00 | 4.02 |
| p-Value | p < 0.02 | NS | P < 0.02 | P < 0.01 | P < 0.005 | P < 0.01 | P < 0.01 |

TABLE 3
Effects of dichloroacetate (DCA) in the presence of 0.48 nM insulin on glucose utilization,
lactate production, the percentage of glucose carbon taken up that was released as lactate,
and leptin production by isolated rat adipocytes over 96 hours in culture.

| DCA (mM) + | | Lactate | | Leptin (ng) | % ΔLeptin | [Leptin] | % Δ[Leptin] |
|---|---|---|---|---|---|---|---|
| Insulin (0.48 | Glucose Uptake | Produced (μg) | Glucose to | Produced | Produced | (ng/ml) | (ng/ml) |
| nM) | (μg) over 96 h | over 96 h | Lactate (%) | over 96 h | over 96 h | at 96 h | at 96 h |
| Control (n = 6) | 1129 ± 92 | 263 ± 9 | 24.1 ± 2.0 | 75.4 ± 6.3 | — | 22.3 ± 1.8 | — |
| 2.0 (n = 6) | 1082 ± 117 | 81 ± 4 | 7.9 ± 0.9 | 88.3 ± 7.5 | — | 27.0 ± 2.6 | — |
| Change in | −46 ± 57 | −181 ± 10 | −16.1 ± 1.3 | +12.9 ± 3.6 | +17.6 ± 4.7 | +3.6 ± 1.0 | +17.9 ± 7.9 |
| Parameter | | | | | | | |
| t-value | NS | 18.10 | 12.38 | 3.58 | 3.74 | 3.60 | 2.27 |
| p-Value | p < 0.01 | P < 0.0001 | P < 0.0001 | P < 0.01 | P < 0.01 | P < 0.01 | P < 0.05 |

Additional experiments tested the effects of DTNB and DCA on glucose oxidation as assessed by the incorporation of radiolabeled glucose carbon into CO₂. In these experiments, DTNB and DCA increased glucose oxidation by isolated adipocytes (FIGS. 13A and 13B). In addition, to its effects on diverting glucose away from anaerobic metabolism to lactate, DCA also decreases the concentration of lactate present at the start of the incubations, showing that DCA promotes the conversion of lactate to pyruvate. This is a result of increased lactate to pyruvate flux through an isoform of lactate dehydrogenase that coverts lactate to pyruvate. Therefore, a method to increase lactate metabolism to pyruvate would also enhance leptin production.

Role of Malic Enzyme and NADPH:

In the fed state (i.e. high insulin and increased glucose flux), the pyruvate-malate cycle serves to transport acetyl-CoA from the mitochondria to the cytosol and to generate NADPH via the action of malic enzyme (see FIG. 4). Acetyl-CoA units are transported from the mitochondria in the form of citrate via a tricarboxylic acid carrier. Citrate stimulates leptin secretion in the presence of low glucose and insulin concentration (Rudolph et al, 1997), whereas in a situation when citrate flux out of the mitochondria is already increased (presence of high insulin and glucose), citrate does not affect leptin secretion. Therefore, citrate could either enter the mitochondria for oxidation in the TCA cycle, or be cleaved by citrate lyase with the OAA generated being converted to malate (via malate dehydrogenase) and then to pyruvate via malic enzyme. That the flux of substrate through malic enzyme may be important in regulating leptin production is suggested by the results of several experiments. First, in addition to inhibiting PDHK, DCA is known to stimulate malic enzyme activity (Mann, 1992) and this action might be involved in its effect to increase leptin secretion (see above). Since concentrations of DCA from 0.1 to 5.0 mM all markedly lowered lactate production to a similar extent, but 2.0 mM was the most potent in stimulating leptin secretion, DCA may increase leptin secretion by another mechanism in addition to inhibition of PDHK, and this could be by activating malic enzyme. Second, the addition of exogenous malate to the culture system of the present invention modestly stimulates leptin production (+20%) in the presence of low insulin and glucose (FIG. 11A). Third, fumarate, which is known to an allosteric activator of malic enzyme (Moreadith, 1984) also increases leptin secretion (+20%), and enhances the stimulation of leptin secretion by malate to approximately +50% over control (FIG. 11A), suggesting that increased flux into the malate-pyruvate cycle is a regulator of leptin production. Thus, the effects of citrate and malate to stimulate leptin in the presence of low glucose provide further support for a role for mitochondrial metabolism and the pyruvate-malate cycle in the effects of glucose metabolism to increase leptin production. An increase of NADPH by malic enzyme may be a cytosolic signal of increased energy flux into mitochondrial metabolism

Role of Fat Oxidation:

The results of three different experiments suggest that increases of fatty acid oxidation may stimulate leptin production. As discussed above a moderate concentration of the uncoupling agent, DNP, modestly increased glucose oxidation (FIG. 5B) and leptin secretion (FIG. 9D). Experiments were also carried out to examine fatty acid oxidation by measuring the incorporation of ¹⁴C-labeled oleate into CO₂. Thus, uncoupling with DNP increases both carbohydrate and lipid oxidation (FIGS. 9B and 9C), perhaps as a compensatory mechanism to produce energy from any available substrate when ATP production is suppressed. Therefore, to further examine the potential role of fatty acid oxidation adipocytes were incubated with L-carnitine, a cofactor of the rate-limiting step for fatty acid transport into the mitochondria via carnitine-palmitoyl-tranferase. (CPT). Carnitine treatment increased fatty acid oxidation (FIG. 10A), inhibited glucose utilization, glucose oxidation, and glucose incorporation into lipid (data not shown), and modestly increased leptin secretion (FIG. 10B). These results provide additional evidence that lipid oxidation, in addition to glucose oxidation, can increase leptin production. Lastly, the addition of oleic acid (2 mM) in the presence of low glucose inhibits glucose oxidation and increases leptin secretion (FIG. 11B).

Role of Energy and Redox Potential (ATP, NADH, and NADPH):

The redox potential of the adipocyte is another mechanism by which substrate metabolism could lead to increased leptin production. In glycolysis, NADH is formed at the glyceraldehyde 3-phosphate dehydrogenase (G-3-P-DH) step. If the pyruvate formed at the end of glycolysis is anaerobically metabolized to lactate, NADH is taken to NAD and there is no net increase of NADH or the NADH/NAD ratio. The formation of lactate allows glycolysis to continue under anaerobic conditions since NAD is reformed and the flux through G-3-P-DH can continue. Without the reformation of NAD, glycolysis would back up and no glucose would be utilized. If the pyruvate from glycolysis is metabolized via PDH and enters the mitochondria then NAD in the cytosol needs to be regenerated via the malate/aspartate or glycerol phosphate shuttle systems in order for glycolysis to continue.

The pyruvate-malate cycle plays a role in mediating insulin-induced leptin secretion. A key step in this cycle is the conversion of malate to pyruvate via malic enzyme (FIG. 4). Malate and its allosteric activator increase leptin secretion. Activation of malic enzyme could contribute to the effect of DCA to increase leptin secretion (FIG. 8D). Pyruvate in the absence of insulin and glucose stimulates leptin secretion. However, the present invention shows that in the presence of glucose and insulin pyruvate actually inhibits leptin secretion. Thus pyruvate may be exerting an end-product inhibition of malic enzyme and thereby reducing flux through the pyruvate-malate cycle. This is similar to the effects of citrate and malate to stimulate leptin secretion in the presence of low, but not higher, levels of insulin and glucose. The conversion of malate to pyruvate via malic enzyme generates NADPH. NADPH is an important contributor to the cellular redox state and in addition supplies reducing energy used in fatty acid synthesis. Although NADPH can also be produced via the pentose phosphate pathway, the production of NADPH from that pathway is coupled to fatty acid synthesis and NADPH is used as lipogenesis proceeds. In contrast the NADPH generated by malic enzyme is not necessarily used for lipogenic purposes and therefore may serve as a signal of cellular energy surplus, which is the condition under which leptin production is increased in adipose tissue.

### PDHK Inhibitors/Adiponectin

Adiponectin (30 kDa) is a secreted protein expressed exclusively in differentiated adipocytes. Primary sequence analysis reveals four main domains: a cleaved amino-terminal signal sequence, a region without homology to known proteins, a collagen-like region, and a globular segment at the carboxy terminus. The globular domain forms homotrimers, and additional interactions between adiponectin collagenous segments cause the protein to form higher order structures. Adiponectin was cloned in 1995/96 and is also known as AdipoQ and Acrp30, and its human homologue has been designated independently as apM1 and GBP28.

In US patent application 20030147855 to Zolotukhin, et al. published Aug. 7, 2003 it was indicated that adiponectin cDNA was cloned into AAV serotypes 1, 2, and 5-based expression vectors. Virions containing these vectors were administered to the livers of rat subjects via portal vein injection. A single injection of 6×10¹¹ virions of the vector caused a sustained and statistically significant reduction in body weight of the treated animals compared to the control animals. This occurred in the absence of side effects. Compared to control animals, the subject rats also exhibited reduced adipose tissue mass, reduced appetite, improved insulin sensitivity, and improved glucose tolerance.

FIG. 3 is a schematic diagram of an important mechanism in the action of insulin to increase the flux of glucose carbon into the mitochondria for oxidative metabolism is activation of pyruvate dehydrogenase (PDH). The activity of PDH is decreased when it is phosphorylated and increased when it is in the dephosphorylated state. Insulin increases flux through PDH by activating PDH phosphatase. PDH kinase (PDH-K) inhibits the activity of PDH by phosphorylating the PDH enzyme complex as shown in FIG. 3.

Three compounds were tested for their ability to inhibit the activity of PDH-K in an adipocyte culture system. Two PDH-K inhibitors that inactivate PDH-K by thiol-disulfide exchange (Pettit, 1982), N-ethylmaleimide (NEM 0.1 μM) (n=6) and 5,5′-Dithiobis(2-nitrobenzoate) (DTNB 100 μM) (n=11), increased adipocyte glucose utilization by 30-80% (FIG. 16A), decreased anaerobic metabolism to lactate (FIG. 16B), increased the amount of glucose not being metabolized to TG or lactate (FIG. 16C) and increased leptin production (FIG. 16D). None of the three compounds increased the proportion of glucose incorporated into TG. DCA and DTNB increased both absolute and proportional glucose oxidation as determined by incorporation of labeled glucose into CO₂ (FIGS. 16E & 16F, n=6). Effects of inhibitors are represented as percent of control values (*p<0.05).

The results with biochemical inhibitors of the PDH regulatory enzyme, PDH-K, show that PDH is a critical control point in the metabolic regulation of leptin. A small molecule drug can be used to inactivate PDH-K in cultured adipocytes. The insertion of antisense oligonucleotides represents another approach (Stein, 1999; Myers, 2000). Primary adipocytes were transfected with an oligonucleotide designed to have an antisense sequence to DNA coding for PDH-K 2 and 4, or with a nonsense oligonucleotide. An adenovirus-assisted DNA transfer method was used to translocate the antisense or nonsense oligos into cultured adipocytes. In the antisense transfected cells a highly significant decrease (35%) in anaerobic glucose metabolism was observed as shown in FIG. 17A and increase (80%) in leptin secretion (FIG. 17B) (n=7). This experiment corroborates results from the biochemical studies with PDH-K inhibitors.

Several studies have reported that thiazolidenediones (TZDs), agonists of PPARγ, increase adiponectin expression and circulating levels in animals (Maeda, 2001; Yamauchi, 2001a; Ye, 2003). TZD (10 μM Troglitazone) stimulated of adiponectin secretion from cultured rat adipocytes from 3 different depots (n=6). Mesenteric fat produced the largest amount of adiponectin over 96 hours in culture. (FIG. 18A). Adiponectin secretion from both the control- and TZD-treated adipocytes was well correlated with the glucose utilization (control, r=0.79; p<0.001); (TZD-treated, r=0.84, p=<0.0001, FIG. 18B) showing that, similar to leptin production, glucose metabolism also is involved in the regulation of adiponectin production by adipocytes.

Incubation of adipocytes with 1.6 nM insulin induced a significant increase in adiponectin secretion during 96 hour culture which was significant after 48 hours (96 hr total 179.9±35.3 vs 312.3±44 ng, n=6, p=0.0005, FIG. 19A). Insulin increased adiponectin production from 3 different fat depots, however as in the experiments with troglitazone, the mesenteric depot produced the most adiponectin (FIG. 19B). Like leptin, both basal and insulin-stimulated adiponectin secretion was highly correlated to glucose utilization (r=0.91, p<0.0001) (FIG. 19C), and inversely related to the proportion of glucose metabolized to lactate (r=−0.81, p<0.0001) (FIG. 19D).

Also paralleling the regulation of leptin, adiponectin secretion by isolated cultured adipocytes was increased during 96 h culture by inhibitors of PDH-K. DTNB (100 μM) increased adiponectin secretion by 40% (p<0.03, n=6, FIG. 20A). DCA (2 mM) increased adiponectin secretion by 23% (p<0.025, n=6, FIG. 20B). The increase in adiponectin induced by DTNB was highly correlated with glucose utilization (r=0.95, p<0.004). Furthermore in adipocytes transfected with antisense directed at PDH-K, adiponectin secretion was stimulated by 24.3±8.4%, p<0.05, n=7, FIG. 20C).

The data show that there are important parallels in the regulation of the adipocyte hormones leptin and adiponectin. The production of both hormones is increased by insulin, positively linked with aerobic glucose metabolism, and inversely related to anaerobic glucose metabolism. The production of both hormones is increased by incubation of isolated adipocytes with biochemical inhibitors of PDH kinase or incorporation of antisense oligonucleotides directed to PDH kinase. The use of PDH kinase inhibitors to increase the production of leptin is shown above as in FIGS. 8A to 8D. The results per FIGS. 16-20 show that PDH kinase is also a promising target for increasing the production of adiponectin. These results will allow those skilled in the art to identify other potent inhibitors of PDH kinase to stimulate adiponectin production in vivo for the treatment of obesity, the metabolic (insulin resistance) syndrome, dyslipidemia, diabetes mellitus, hepatic steatosis, and cardiovascular disease.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Materials and Methods for Adipocyte Culture

Materials: Media (DMEM) and fetal bovine serum (FBS) are purchased from Life Technologies (Grand Island, N.Y.). The media is supplemented with 6 ml each of MEM nonessential amino acids, penicillin/streptomycin (5000 U/ml/5000 ug/ml), and nystatin (10,000 U/ml; all from Life Technologies) per 500 ml DMEM. Bovine serum albumin (BSA) fraction V, HEPES, collagenase (Clostridium histolyticum; type II, SA 456 U/mg), insulin, NEM, and DTNB are purchased from Sigma Chemical Co (St. Louis, Mo.). Matrigel matrix is purchased form Becton Dickinson (Franklin Lakes, N.J. Collagen is purchased from Cohesion Technologies, (Palo Alto, Calif.). Nylon filters are purchased from Tetko (Kansas City, Mo.).

Animals: Results were obtained using isolated rat adipocytes. However, techniques described here can be conducted in isolated mouse adipocytes. (Gregoire F, Stanhope K L, Havel P J, West D B. Functional assessment of insulin-stimulated glucose utilization in cultured adipocytes derived from C57BL/6J and DBA/2J inbred mice. Obesity Res. 8 (Suppl. 1): 66S, 2000). Male Sprague-Dawley rats (3-6 months of age) are obtained from Charles River (Wilmington, Mass.) or Harlan Sprague-Dawley. Animals are housed in hanging wire cages in temperature controlled rooms (22° C.) with a 12-h light-dark cycle and fed Purina chow diet (Ralston-Purina, St. Louise, Mo.) and given deionized water ad libitum. Animal use and care is in accordance with the National Institutes of Health Guide for the Use and Care of Laboratory Animals and conducted in facilities accredited by the American Association for Accreditation of Laboratory Animal Care (AAALAC). The study protocols have been approved to the Administrative Animal Use and Care Committee at the University of California, Davis.

Methods:

Cell isolation/preparation: Adipocytes are prepared from epididymal fat pads from male Sprague-Dawley rats weighing 300-600 g. Epididymal fat depots are resected from halothane anesthetized rats under aseptic conditions and adipocytes are isolated by collagenase digestion by the Rodbell method (Rodbell M. Metabolism of isolated fat cells. I. Effects of hormones on glucose metabolism and lipolysis. J Biol Chem. 1964; 239: 375-380), with minor modifications as previously described (Mueller W M, Gregoire F M, Stanhope K L, Mobbs C V, Mizuno T M, Warden C H, Stern J S, Havel P J. Evidence that glucose metabolism regulates leptin secretion from isolated adipocytes. Endocrinology 139: 551-558, 1998; Mueller W M, Stanhope K L, Gregoire F, Evans J L, Havel P J. Effects of metformin and vanadium on leptin secretion from cultured rat adipocytes. Obesity Res. 8: 530-539, 2000; Medina E A, Stanhope K L, Mizuno T M, Mobbs C V, Gregoire F, Hubbard N E, Erickson K L, Havel P J. Effects of tumor necrosis factor alpha on leptin secretion and gene expression: relationship to changes of glucose metabolism in isolated rat adipocytes. Int J Obes Relat Metab Disord. 23: 896-903, 1999.). The isolated adipocytes are then incubated for 30 minutes at 37 C before being plated and cultured on Matrigel-coated plates.

Adipocyte Culture: Adipocytes are maintained in culture anchored to a basement membrane matrix (Matrigel, Becton Dickinson, Franklin Lakes, N.J.) or collagen from Cohesion Technologies, (Palo Alto, Calif.). Although all in vitro systems have inherent advantages and disadvantages, advantages of this system compared with cultures containing free-floating adipocytes are that the matrix simulates their normal basement membrane attachment and that the cells are maintained in close proximity to each other, allowing direct cell to cell contact. Together the cell contact and basement membrane attachment help to maintain differentiation, since adipocytes have a strong tendency to dedifferentiate in long-term (>24 h) culture. In addition, the matrix and the small amount of serum in the media both contain growth factors, which are also likely to help in maintaining cell differentiation. Furthermore, the adipocytes in this system are not exposed to toxic levels of oxygen at the interface of the media and the incubator atmosphere, as opposed to free-floating adipocytes which aggregate at the surface of the media. An advantage of the system over those containing minced adipose tissue is that all of the cells in the culture are equally exposed to the nutrients and the oxygen dissolved in the media. Thus, although clearly different from the in vivo situation, this system provides a more physiological environment than most systems for maintaining adipocytes in long-term culture.

The goal of these experiments was to examine the direct actions of metformin and vanadium on leptin production and adipocyte metabolism. Therefore, the advantage of employing in vitro experimentation for this purpose over in vivo models is that it was possible to control confounding variables, such as effects of these agents on food intake (Havel P J. Mechanisms regulating leptin production: implications for control of to energy balance. Am J Clin Nutr. 1999; 70:305-306; Havel P J. Role of adipose tissue in body-weight regulation: mechanisms regulating leptin production and energy balance Proc. Nutr. Soc. 59: 359-371, 2000), which would indirectly influence leptin production via changes of insulin secretion (Saad M F, Khan A, Sharma A, et al. Physiological insulinemia acutely modulates plasma leptin. Diabetes. 1998; 47: 544-549; Havel P J, Townsend R, Chaump L, Teff K. High fat meals reduce 24 hour circulating leptin concentrations in women, Diabetes. 1999; 48:334-341). Unlike an in vivo system, in these experiments the environment surrounding the adipocytes within the individual wells of each culture plate was identical with the exception of the presence or absence and the concentration of metformin or vanadium, allowing assessment of the direct effects of the treatments.

In culturing each suspension, Matrigel is first thawed on ice to a liquid and uniformly applied to the surface of culture dishes (300 μl Matrigel/35 mm well). After the incubation period, 150 μl of the adipocyte suspension (2:1 ratio of packed cells to media) are plated on the Matrigel or collagen martix. Adipocytes from each suspension are thoroughly mixed with a transfer pipette before plating to insure that a similar number adipocytes with a similar size distribution are added to the control and experimental wells for each suspension. The warmth of the added cells and buffer causes the Matrigel to gel around the adipocytes, or the neutralization of the acidic pH of the collagen solution to ˜7.0 solidifies the collgen, and both of these techniques effectively anchor the adipocytes to the culture dish. After a 30 minute incubation at 37° C., 2 ml of warm culture medium is added. The cells are maintained in an incubator at 37° C. for 96 hours with 6% CO₂. Aliquots of adipocytes from each animal are divided into wells, with the different concentrations of insulin or other agents to be tested. In each plate an appropriate control well contains adipocytes from the same animal. Adipocytes are incubated with media (DMEM) containing 5.5 mM (100 mg/dl) glucose plus 5% FBS at several concentrations of inhibitors to be tested. In all experiments, aliquots of media, 300 μl, (15% of the media volume) is collected from culture wells and replaced with fresh media containing the appropriate concentrations of insulin or other agents to be tested at 24, 48, 72, and 96 hours.

Incorporation of Glucose Carbon into Triglyceride: To measure glucose incorporation into triglyceride, cultures are exposed to media containing 0.01 uCi/ml of ¹⁴C-glucose. After 96 hr, media and extracellular lipid is removed from the well and methanol added. Then scrape the collagen-cell matrix from the well and transfer into a 50 ml glass tube. Rinse the well and scrape again in methanol to assure complete transfer of cells. Total triglycerides will be extracted using the Folch method (Folch, 1957). An aliquot of the lipid extract will be placed into vials containing scintillation fluid then radioactivity will be measured.

The first measurement is used to calculate the amount of glucose incorporated into triglycerides. Another aliquot of the lipid extract is placed into pre-weighed aluminum pans to determine the total amount of triglyceride per well. The remaining lipid is saponified and acidified to separate the glycerol and fatty acids. An aliquot of the lipid extract is placed into vials containing scintillation fluid and counted. This second count represents the ¹⁴C-glucose incorporation into fatty acids. By subtraction, the amount that was incorporated into triglyceride though glycerol is also determined. Glucose incorporation into triglyceride and into the fatty acid portion of the triglyceride are calculated by multiplying disintegration per min by total ug of glucose/well over the total DPM/well.

Substrate Oxidation: Oxidation is measured using a modification of the method of Rodbell (Rodbell, 1964) and a modification of the cell culture system described by Bottcher and Furst (Bottcher, 1996). Briefly, adipocytes are isolated, counted and sized as previously described. Adipocytes are plated as described except they are placed in a sterile 20 ml scintillation vial instead of a well. Two ml of treatment media containing [U-¹⁴C]-substrate (0.3 uCi/ml; glucose, fatty acids, malate, fumarate, pyruvate) is added to the vials. The vials are filled with 95% O₂-5% CO₂ gas and capped with rubber stoppers fitted with a hanging center well. Each well contains a 2×8 cm strip of Whatman No. 1 paper. Vials are maintained at 37° C. for 48 hr. After 48 hr, a sample of media is removed from each vial using a 4 inch, 23 gauge needle. Using another syringe and 23 gauge needle, 200 ul of sodium benzethonium is placed onto the paper strip and hanging well to capture CO₂. Concentrated sulfuric acid is added to the vial in order to lyse cells and liberate all CO₂ from the collagen matrix. After 24 hours, the hanging well and paper are transferred to another vial containing scintillation fluid and counted. The data are expressed as % DPM recovered as CO₂ of the total DPM remaining in the media at 48 hours and as micromoles of substrate oxidized over time.

Northern Blot Procedure: RNA is extracted according to the Gibco Life Technologies procedure using Trizol (Life Technologies Inc., Grand Island, N.Y.). UV absorbance and integrity gels is used to estimate RNA. The cDNA probe for leptin has been kindly provided by Dr. Charles Mobbs (Mount Sinai School of Medicine, New York). The cDNA probes for malic enzyme; CPT and PDH are purchased from Molecular Probes, Eugene, Oreg. cDNA probes are labeled by random priming (Rediprime kit, Amersham) in the presence of ³²P dCTP (3000 Ci/mmol, Amersham). Unincorporated nucleotides are removed using NucTrap probe purification columns (Stratagene, La Jolla, Calif.). For each tissue sample, 5-10 μg of total RNA is fractionated by electrophoresis on a denaturing 1% agarose gel containing 2.2 M formaldehyde and 1×MOPS running buffer. One μl of a 50 μg/ml ethidium bromide stock solution is added in order to check RNA integrity and even loading. After electrophoresis, RNA is transferred onto a nylon membrane (Duralon-UV, Stratagene, La Jolla, Calif.) by overnight capillary transfer and UV cross-linked (Stratalinker 1800, Stratagene, La Jolla, Calif.). Blots are hybridized for 1 hr at 68° C. in presence of the labeled cDNA probe (2×10⁶ cpm/ml Express hyb solution). Blots are washed 2× at high stringency and exposed to X-ray films with an intensifying screen for 2 days at −80° C. (Kodak BioMax). Leptin mRNA is analyzed using a single-stranded cDNA probe and quantified using a phosphoimager. Blots are analyzed again using a probe complementary to mouse 18S ribosomal RNA. mRNA levels are normalized with respect to the 18S ribosomal RNA signal.

Assays: Leptin concentrations in the medium are determined with a sensitive and specific RIA for rat leptin (Landt M, Gingerich R L, Havel P J, Mueller W M, Schoner B, Hale J E, Heiman M L. Radioimmunoassay of rat leptin: Sexual dimorphism reversed from humans. Clin Chem. 1998; 44:565-570) or for mouse leptin (Ahren B, Mansson S, Gingerich R L, Havel P J. Regulation of plasma leptin in mice: influence of age, high-fat diet, and fasting. Am. J. Physiol. 273: R113-120, 1997) with reagents obtained from Linco Research, St. Charles, Mo. Glucose and lactate are measured with a YSI glucose analyzer (Model 2300, Yellow Springs Ins., Yellow Springs, Ohio).

Data Analysis: The uptake of glucose is assessed by measuring the concentration of glucose in the media in each well before and at 24, 48, 72, and 96 hours of incubation and calculating the decrease over 96 hours, after correcting for the amount of glucose that was removed during each 24 h media sampling and the amount added by the replacement of fresh media (15% of total volume). Lactate production is calculated as the increase of media lactate at 24, 48, 72, and 96 hours, correcting for the amount of lactate removed by sampling and added with media replacement. To examine the relationship between adipocyte carbon flux and leptin secretion in adipocytes, the amount of carbon released as lactate per amount of carbon taken up as glucose over 96 hours is calculated as lactate production/glucose utilization, and expressed as a percentage. Cumulative leptin production is calculated as the change of media leptin concentrations at 24, 28, 72, and 96 hours, correcting for the amount of leptin removed during sampling. The area under the curve for leptin production between 0-96 hours is calculated by the trapezoidal method. The experimental results from each adipocyte suspension prepared from a single animal are analyzed in relation to a control well from the same suspension. To examine the relationships between glucose uptake, lactate production, glucose conversion to lactate, and leptin secretion, simple and multiple linear regression analyses are performed with a statistics software package (StatView for Macintosh, Abacus Concepts, Inc., Berkeley, Calif.). Data are expressed as means±SEM.

### Example 2

### Adipocyte Culture Protocol

Day Before Preparation:

Make phosphate-hepes buffer (instructions on folsh dessicator).

Autoclave supplies: Incubation jars (60 ml for rat, 30 ml for mice), filters (400 um for rat, 250 um for mice) long needles (+6), 1 ml pipet tips (+6 boxes), 0.2 ml pipet tips (1 box), surgical equipment (3-5 small scissors, 3 large scissors, 3 forceps), 500 ml reagent jars, 250 and 100 ml reagent jars.

Cut long needle plastic covers to sterilize under uv if needed.

Clear and clean hood, turn on uv light.

Media Preparation:

Place buffer in incubator to warm.

Place 6 ml tubes of FBS, nystatin, penicillin (all in FC freezer) in incubator to thaw.

Get 500 ml bottle of DMEM from walkin cold room (check glucose content).

Place microfuge tube of insulin stock in hood to thaw (−80 freezer, 2^{nd} shelf, FC insulin box).

Place microfuge tube of C14 glucose stock in hood to thaw (FC freezer, FC C14 glucose stock).

Turn on hood light in order to turn off uv.

Make basic media by adding 6 ml of FBS, nystatin, penicillin and nonessential amino acids (in FC refrigerator) to 500 ml DMEM.

Make medias (can be the most difficult, intensive, and time-consuming part).

Prepare insulin.

Dilute insulin stock 10×s (0.1 ml to 1.0 ml)

Sterilize with 0.2 um syringe filter.

Label it 160 nM insulin stock.

Dilute 160 nM insulin stock 100×s (0.1 ml to 10 mls).

Label it 1.6 nM insulin stock.

Mix well.

Dilute 1.6 nM insulin stock to 0.48 nM stock and label (1.5 ml to 5 mls).

Dilute 1.6 nM insulin stock to 0.16 nM stock and label (1 ml to 10 mls).

Add the appropriate amount of insulin to medias.

10 microlites of the insulin stock added to 1 ml of media=conc of stock label insulin media (i.e. 100 microliters of 1.6 nM insulin stock to 10 ml of media=1.6 nM insulin media).

Mix medias well, loosen lids, and store in incubator until needed. Place extra DMEM in incubator until needed.

Prepare for Harvesting Adipocytes

Cut lab covering for each carcass and label with animal #, absorbent side up.

Label two 60 mm culture dishes with animal # for each animal.

Place 1 dry set by microbalance.

Add buffer to other set.

Place surgical equipment in beaker with 70% EtOH.

Fill a 15 ml labeled conical with buffer.

Label one 10+ ml edta purple top vacutainer with rat #

Set aside lid and place small plastic funnel in tubes.

Get ice for bloods.

Turn on water bath to 37 degrees.

Set up and place FC notebook by microbalance.

Prepare collagenase (5 gram dry-bottle in FC refrigerator).

Rat collagenase concentration=1.25 mg/ml.

Need 2 ml/gram of fat

Need 4 grams of fat/suspension

Therefore for each rat weigh out 10 mg of dry collagenase.

Transfer to 50 ml conical tube.

Add 8 ml buffer/10 mg dry collagenase

(Standard 6 rat recipe=60 mg collagenase/48 ml of buffer)

Mix collagenase well and sterilize with steriflip.

Store in incubator until needed.

Mice collagenase concentration=0.625 mg/ml

Need 2 ml/gram of fat

Assume less than 1 gram of fat/mouse.

Transfer to 50 ml conical tube.

Add 8 ml buffer/5 mg dry collagenase

(Standard 6-10 mouse recipe=12.5 mg collagenase/20 ml of buffer)

Mix collagenase well and sterilize with steriflip.

Store in incubator until needed.

Ready to harvest adipocytes:

Add halothane to harvest adipocytes jar.

Place animal in harvest adipocytes jar.

When unconscious, weigh and record.

Deccapitate, and collect truncal blood in funnel and tube.

Place lid on blood tube, invert, store on ice until centrifuging and separating is possible.

Place animal on harvest adipocytes cloth and take to hood.

Fat Digestion:

Remove epididymal fat pad using buffer-rinsed surgical equipment and place in labeled culture dish with buffer.

Tare dry labeled culture dish with micro balance.

Under hood, transfer epi pad to culture dish using buffer-rinsed forceps.

Weigh and record.

If fat pad weighs more than 4-4.5 grams, remove extra fat using buffer rinsed scissors.

Record suspension fat pad weight on culture dish and in book.

Bring pad back to hood, and re-add buffer.

When all animal fat pads are weighed, add 2 ml of collagenase/gram of fat to labeled suspension jars.

Transfer fat pad to lid of culture dish.

Set timer.

Mince fat for 1-2 minutes (one minute when experienced, two when novice).

Using cell scraper, transfer minced fat to incubation jar.

Set timer and record incubation start time on lid of jar.

Parafilm lid of jar.

Place in 37 degree shaking (motor on 6) water bath for 30 minutes.

Place buffer in incubator

Fat Cleaning:

During incubation prepare for filtration.

For each rat, label a 50 ml conical.

Remove lid and place a 400 um filter on top of tube.

Use a 25 ml pipet to force filter into tube.

For each mouse label a 15 ml conical.

Remove lid and place a 250 um filter on top of tube.

Use a 10 ml pipet to force filter into tube.

At 30 minute incubation (+/−only 1 minute) remove suspension jar from bath.

Add 24 ml buffer (10 ml for mice) and pipet up and down 4 times to mix.

Transfer suspension to filter, making sure pipet is in filter, not in a fold.

Allow suspension to drain.

Making sure gloved hands are sterile, scrape filter into conical tube.

Add buffer up to 40 mls (14 ml for mice).

Centrifuge at 1000-1100 rpms—check setting—for 6 minutes.

During centrifuging prepare syringes for cleaning steps.

For each animal label a 20 ml syringe.

Place a long autoclaved needle on syringe.

Place a plastic cover on needle.

Place syringes with needles upright—3 to a 600 ml beaker—to keep sterile.

Label a 600 ml beaker for waste.

At end of centrifuge remove the buffer from underneath cell layer with needle and syringe.

Place this buffer in waste beaker.

Add fresh buffer to 25-35 ml (10-14 ml for mice) depending on quantity available.

Centrifuge at 1000-1100 rpms for 6 minutes.

At end of centrifuge remove buffer and replace with 8-10 ml basic media.

Transfer to labeled 15 ml conical by pouring.

Centrifuge at 1000-1100 rpms for 6 minutes.

At end of centrifuge remove media and add fresh up to no more than 14 ml.

Place in incubator and start a timer.

Incubate for at least 30 minutes, but less than an hour.

Plating in 6 Well Plates and Oxidation Vials:

During incubation prepare collagens

Calculate the amount needed figuring 0.5 ml/well and 0.3 ml/oxidation vial plus an extra 3-5 ml.

Transfer that amount to an appropriate-size sterile container (15 or 50 ml conical, 100 ml reagent bottle, or collagen bottle). To minimize collagen waste, pouring is better than pipeting.

Add 1 ml 10×DMEM (50 ml conical tubes in door of FC refrigerator) per 10 ml collagen.

Added 10 M NaOH to collagen to get pH=7, using a red color to judge (not orange, not pink).

It is usually safe to add 0.5% initially (50 ul/10 ml collagen), but collagen can vary by lot and this “safe” quantity can change.

After initial 0.5%, added NaOH only 1-5 ul at a time.

Try not to overshoot since this seems to affect ability of collagen to set.

Try not to take too long, as the collagen can start setting during this process.

When regular collagen is red and ready, add C14 glucose to the appropriate amounts at 1 ul/ml for lipogenesis work (label 1× collagen), and at 3 ul/ml for oxidation work (label 3× collagen).

Set up for collagen pipeting by having a 1 ml pipet for each type of collagen (usually 3—for regular, 1×, and 3×).

Have a 50 ml conical tube labeled to hold each pipets and keep sterile (minimizes the need for fresh tips with each pipeting).

Prepare plating plan based on amount of fat in suspensions, and culture objectives and priorities.

At end of incubation and when collagen, vials, plates are ready, prepare susp 1 for plating.

Remove media to a 2 fat to 1 media ratio.

Use an accurate 200 ul pipet with a sterile wide-open tip for fat pipeting.

To conserve fat, try to complete all pipeting from a single suspension using the same tip.

Mix initially by inverting and then with pipeting, such that suspension is homogenous immediately before each well and vial is plated.

Collagen pipetor person places 0.5 ml collagen in a well, or 0.3 ml collagen in a vial.

Fat pipetor person adds 150 ul of fat suspension directly on collagen.

Plates are gently moved in a circular motion on level surface to spread collagen over entire surface.

Place plated plates and vials in incubator immediately.

Collagen in vials must be in contact with metal shelf to set (use a vial separator insert to avoid tipping).

Finish the plating for all suspensions.

When plating is finished and collagen has set, add 2 ml of appropriate media to each well and label.

Make sure each plate is labeled with FC # and return to incubator.

Place vials by suspension in styrofoam 50 ml conical racks and label (FC # too).

Add 2 ml of media without touching inside of vial.

Return vials to incubator.

While vials are still in incubator, set rubber stopper (with wells and Whatman 1 filter strips) on oxidation vials only 6-10 at a time, when the incubator CO2 is no less than 5%.

Remove rack of vials from incubator and using 2 people, secure rubber stoppers.

Media must not touch wells and paper strips.

Suspension for Sizing and Lipid Measurement:

There must be at least one well/suspension earmarked for sizing and lipid measurement on regular collagen.

Add 2 ml basic media to these wells.

Take 3 Image Pro pictures of each suspension.

Between each suspension take a picture of the suspension #, using the numbered culture lid.

When pictures are taken, aspirate off the 2 ml of media removing as much of the extracellular lipid as possible.

Add 4 ml of methanol to each well.

Parafilm the plate, and replace lid.

Place in refrigerator, making sure each plate is labeled with FC #.

End of 0 Hour Day.

### Examples 3 and 4

### Leptin Production Enhanced Via Nucleotide Sequences

Methods and Materials

Identification and synthesis of PDH-K active site antisense oligonucleotide candidates and nonsense oligonucleotide: The 5 prime end of the PDH-K gene was targeted for possible active site sequences. Net Primer 3 and other similar computer modules was used to confirm and disqualify candidates as primers, based on melting point, % GC content, and tertiary structure. Candidate primers were identified or disqualified as a consensus sequences, common to several species, using the NIH BLAST data-base. Candidate sequences for the nonsense oligonucleotide were screened using computer models for confirmation as primer candidate. The NIH BLAST data-base was used to screen candidate nonsense primers as unrelated to metabolic activity. Both oligonucleotides were synthesized by the Molecular Structure Facility of the University of California, Davis.

Transfection of isolated adipocytes with PDH-K active site antisense oligonucleotide and nonsense oligonucleotide sequences: Oligonucleotides were diluted 8 μg/100 μl DMEM. Polyethyleninime (PEI; Aldrich) was diluted 8 μg/200 μl DMEM in polystyrene tubes. Diluted oligonucleotide was added one drop at a time to PEI solution and incubated at room temperature for 15 minutes. A replication-deficient adenovirus was used to assist the transfer of the antisense and “nonsense” oligonucelotides into the cultured adipocytes. Replication-deficient adenovirus (5 dI-342) stock was diluted 2 μl/200 μl DMEM and then added to PEI-oligonucleotide mixture. After 10 minutes of incubation, 250 μl of each Adenovirus/PEI/oligonucleotide mixture were added to duplicate wells of 100 μl of adipocyte suspension. Cells were incubated with mixture for 45 minutes. Transfection media was removed, cells were washed one time, and them 0.3 ml of liquid Matrigel matrix was added. 2 ml of 0.48 nM insulin media were added after Matrigel was set and cells were culture for 96 hours.

Measurement of β-galactosidase activity: At 96 hours, media was removed and the cells were washed 2 time in PBS. 0.4 mls of reporter lysis buffer (Promega) was added to each well and incubated for 15 minutes. Cells and buffer were transferred to microfuge tubes, vortexed, sonicated for 1 second, and centrifuged for 2 min at 12,000 RPM. Lysate was removed and assayed for β-galactosidase activity using Promega β-Galactosidase Enzyme Assay System.

Transfection of isolated adipocytes with adenovirus-malic enzyme construct: Adenovirus-malic enzyme and adenovirus β-galactosidase constructs were obtained. Isolated cells were plated on collagen as previously described. Cells were incubated in transfection media containing adenovirus stock at 37° C. for 24 hours. Media was removed and cells were washed with PBS. Two ml 0.48 nM insulin media was added and cells were cultured for 96 h.

### Example 3

Primary adipocyte cells in cultures were transfected with an oligonucleotide designed to have an antisense sequence to DNA coding for PDH-K. An adenovirus assisted DNA transfer method was used to translocate the antisense oligonucleotide into cultured adipocytes. In these cells (n=7 independent experiments) we observed a substantial decrease of anaerobic glucose metabolism indicated by a highly significant reduction in the proportion of glucose metabolized to lactate (FIG. 14A). In the same cultures, leptin production was markedly increased by an average 82±22% (p<0.01) compared to cells transfected with a control “nonsense” oligonucleotide (FIG. 14B).

The proportional decrease of anaerobic metabolism was highly predictive (r=0.90, p<0.01) of increase of the leptin secretion also observed in this experiment (FIG. 14C). This experiment corroborates the biochemical studies with PDH-K inhibitors and further indicate that a molecular antisense approach is another pharmacological option for increasing endogenous leptin production in vivo. Antisense technology to inactivate specific targets has been suggested in the treatment of a number of diseases including cardiovascular disease (Yla-Herttuala and Lancet. 355: 213-222, 2000), hypertension (Metcalfe et la, Curr Hypertens Rep. 4: 25-31, 2002), and diabetic vascular disease (Serri and Renier, Metabolism. 44: 83-90, 1995).

Adipocytes were incubated with a control (β-Galactosidase) engineered adenovirus and a high degree of transfection was obtained as shown in FIG. 15A. Cultured adipocytes were then transfected with an adenovirus vector engineered to comprise the coding sequences for malic enzyme (MD). Cells incubated with the ME virus secreted 40% more leptin than those incubated with a control (β-Gal) adenovirus (FIG. 15B). This shows that this pathway is involved in the metabolic regulation of leptin production. Furthermore, this experiment shows that a gene therapy approach for increasing leptin production is a useful method for increasing endogenous leptin production in vivo in the treatment of obesity and other conditions in which increased leptin production would be beneficial.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for decreasing fat content, comprising the steps of:
administering an effective amount of a pyruvate dehydrogenase-kinase (PDHK) inhibitor to adipocytes; and
allowing the PDHK to remain in contact with the adipocytes for a period of time and under conditions such that pyruvate dehydrogenase kinase (PDK) is inhibited and fat is decreased.

2. The method of claim 1 wherein the PDH-kinase inhibitor is chosen from 5,5′-Dithiobis(2-nitrobenzoate)(DTNB), Dichloroacetate (DCA), and N-ethylmaleimide (NEM) and the PDHK is administered to tissue comprising adipocytes.

3. The method of claim 1 wherein the PDH-kinase inhibitor is administered to a patient having a body mass index (BMI) of 30 or more.

4. The method of claim 3 wherein the PDH-kinase inhibitor remains in contact with the adipocytes under conditions for a period of time such that the patient's appetite is reduced and the patient is a Type II diabetic.

5. The method of claim 3 wherein the PDH-kinase inhibitor is administered in an oral formulation and the number of adipocytes are reduced and the patient is insulin resistant.

6. The method of claim 3 wherein the PDH-kinase inhibitor is administered in a formulation for parenteral delivery and the fat content of adipocutes is reduced and wherein the adipocytes are human adipocytes

7. A controlled release, oral formulation comprising:
a PDH-kinase inhibitor; and
a pharmaceutically acceptable carrier for administration of an effective amount of PDH-kinase inhibitor to decrease fat in adipocytes or the number of adipocytes;
wherein the PDH-kinase inhibitor inhibits human PDH-kinase.

8. A method of enhancing leptin production, comprising the steps of:
contacting cells with a compound which inhibits pyruvate dehydrogenase kinase; and
allowing the compound to remain in contact with the cells for a period of time and under conditions such that activity of PDHK in the cells is inhibited thereby enhancing production of adiponectin and leptin in the cells.

9. The method of claim 8, wherein the compound is an antisense sequence which reduces PDH-K production in the cells to a level below the level of production prior to contacting the cells with the antisense sequence.

10. A method of enhancing production of adiponectin and leptin, comprising the steps of:
contacting cells with an exogenous nucleotide sequences encoding malic enzyme;
allowing the cells to be transfected with the nucleotide sequences in a manner such that malic enzyme is expressed and adiponectin and leptin production of the cells is enhanced.

11. The method of claim 10, wherein the compound is an antisense sequence which reduces PDH-K production in the cells to a level below the level of production prior to contacting the cells with the antisense sequence.

12. A method of causing adipocytes to enhance production of adiponectin and leptin, comprising
contacting adipocytes with a pyruviate dehydrogenase kinase (PDHK) inhibitor for a period of time and under conditions such that PDHK is inhibited and production of adiponectin and leptin is enhanced.

13. The method of claim 12, wherein the adipocytes are present in an obese human having a BMI of 30 or more.

14. The method of claim 12, further comprising:
transfecting the adipocyte with an antisense sequence which reduces PDHK production.

15. The method of claim 12, further comprising:
transfecting the adipocyte with a sequence encoding malic enzyme.

16. A method for decreasing fat content in a patient, comprising:
administering an effective amount of a compound which elevates pyruvate dehydrogenase-(PDH) activity (PDH elevator) to cells in the patient adipocytes.

17. The method of claim 16 wherein the elevator is administered to a patient once a day or more for three weeks or more and wherein the PDH-elevator reduces appetite.

18. The method of claim 16 wherein the PDH elevator is administered in an oral formulation twice a day or more for four weeks or more and the number of adipocytes in the patient are decreased.

19. The method of claim 16 wherein the PDH elevator is administered in a formulation for parenteral delivery and fat content of adipocytes in the patient is decreased wherein the adipocytes are human adipocytes and the oral formulation is administered for two months or more.

20. The method of claim 19 wherein the adipocytes are in a patient with diabetes.

21. A pharmaceutical composition comprising a PDH elevator and a pharmaceutically acceptable carrier for administration of an effective amount of PDH elevator to decrease fat in adipocytes or the number of adipocytes.

22. The composition of claim 21 wherein the PDH elevator is a controlled release oral formulation.

23. The composition of claim 21 wherein the PDH elevator is chosen from N-(4-benzoyl-2,6-dimethylphenyl)-2-hydroxy-2-methyl-3,3,3-trifluoropropanamide, N-(4-benzoyl-2-methylphenyl)-2-hydroxy-2-methyl-3,3,3-trifluoropropanamide, N-(4-benzoyl-2-fluorophenyl)-2-hydroxy-2-methyl-3,3,3-trifluoropropanamide, N-(6-chloro-3-phenylsulfonylphenyl)-2-hydroxy-2-methyl-3,3,3-trifluoropropanamide, 2-hydroxy-N-[2-methoxy-4-(4-pyridylsulfonyl)phenyl]-2-methyl-3,3,3-trifluoropropanamide, 2-hydroxy-2-methyl-N-[2-nitro-4-(phenylsulfonyl)phenyl]-3,3,3-trifluoropropanamide, S-(−)-N-(4-benzoyl-2-methylphenyl)-2-hydroxy-2-methyl-3,3,3-trifluoropropanamide, N-(4-benzoyl-2-chlorophenyl)-2-hydroxy-2-methyl-3,3,3-trifluoropropanamide, N-(4-benzoyl-2-bromophenyl)-3,3-difluoro-2-(difluoromethyl)-2-hydroxypropanamide, N-(4-benzoyl-2-bromophenyl)-2-hydroxy-2-methyl-3,3,3-trifluoropropanamide, N-(4-benzoyl-2-cyanophenyl)-2-hydroxy-2-methyl-3,3,3-trifluoropropanamide, N-(4-benzoyl-2-methoxyphenyl)-2-hydroxy-2-methyl-3,3,3-trifluoropropanamide, N-(4-benzoyl-2-hydroxyphenyl)-2-hydroxy-2-methyl-3,3,3-trifluoropropanamide, 2-hydroxy-N-[2-hydroxy-4(4-pyridylsulfonyl)phenyl]-2-methyl-3,3,3-trifluoropropanamide, N-(4-benzoyl-2,6-dimethylphenyl)-2-hydroxy-2-methyl-3,3,3-trifluoropropanamide, N-(2-Fluoro-5-nitrophenyl)-2-hydroxy-2-trifluoromethylbutanamide, N-(2-Fluoro-5-nitrophenyl)-2-hydroxy-2-difluoromethyl-3,3-difluoropropanamide, and 3-Hydroxy-3-trifluoromethyl-1-(2-chloro-5-trifluoromethylphenyl)-4,4,4-trifluorobut-1-yne, N-(4-benzoyl-2-methylphenyl)-2-hydroxy-2-methyl-3,3,3-trifluoropropanamide, N-(4-benzoyl-2-fluorophenyl)-2-hydroxy-2-methyl-3,3,3-trifluoropropanamide, 2-hydroxy-N-[2-methoxy-4-(4-pyridyl-sulfonyl)phenyl]-2-methyl-3,3,3-trifluoropropanamide, 2-hydroxy-2-methyl-N-[2-nitro-4(phenyl-sulfonyl)phenyl]-3,3,3-trifluoropropanamide, S-(−)-N-(4-benzoyl-2-methylphenyl)-2-hydroxy-2-methyl-3,3,3-trifluoropropanamide, N-(4-benzoyl-2-chlorophenyl)-2-hydroxy-2-methyl-3,3,3-trifluoropropanamide, N-(4-benzoyl-2-bromophenyl)-3,3-difluoro-2-(difluoromethyl)-2-hydroxypropanamide, N-(4-benzoyl-2-bromophenyl)-2-hydroxy-2-methyl-3,3,3-trifluoropropanamide, N-(4-benzoyl-2-cyanophenyl)-2-hydroxy-2-methyl-3,3,3-trifluoropropanamide, N-(4-benzoyl-2-methoxyphenyl)-2-hydroxy-2-methyl-3,3,3-trifluoropropanamide, N-(4-benzoyl-2-hydroxyphenyl)-2-hydroxy-2-methyl-3,3,3-trifluoropropanamide and 2-hydroxy-N-[2-hydroxy-4-(4-pyridylsulfonyl)phenyl]-2-methyl-3,3,3-trifluoropropanamide N-(4-benzoyl-2-methylphenyl)-2-hydroxy-2-methyl-3,3,3-trifluoropropanamide, N-(4-benzoyl-2-fluorophenyl)-2-hydroxy-2-methyl-3,3,3-trifluoropropanamide, 2-hydroxy-N-[2-methoxy-4-(4-pyridyl-sulfonyl)phenyl]-2-methyl-3,3,3-trifluoropropanamide, 2-hydroxy-2-methyl-N-[2-nitro-4-(phenyl-sulfonyl)phenyl]-3,3,3-trifluoropropanamide, S-(−)-N-(4-benzoyl-2-methylphenyl)-2-hydroxy-2-methyl-3,3,3-trifluoropropanamide, N-(4-benzoyl-2-chlorophenyl)-2-hydroxy-2-methyl-3,3,3-trifluoropropanamide, N-(4-benzoyl-2-bromophenyl)-3,3-difluoro-2-(difluoromethyl)-2-hydroxypropanamide, N-(4-benzoyl-2-bromophenyl)-2-hydroxy-2-methyl-3,3,3-trifluoropropanamide, N-(4-benzoyl-2-cyanophenyl)-2-hydroxy-2-methyl-3,3,3-trifluoropropanamide, N-(4-benzoyl-2-methoxyphenyl)-2-hydroxy-2-methyl-3,3,3-trifluoropropanamide, N-(4-benzoyl-2-hydroxyphenyl)-2-hydroxy-2-methyl-3,3,3-trifluoropropanamide, 2-hydroxy-N-[2-hydroxy-4(4-pyridylsulfonyl)phenyl]-2-methyl-3,3,3-trifluoropropanamide, N-(4-benzoyl-2-methylphenyl)-2-hydroxy-2-methyl-3,3,3-trifluoropropanamide, N-(4-benzoyll-2-fluorophenyl)-2-hydroxy-2-methyl-3,3,3-trifluoropropanamide, 2-hydroxy-N-[2-methoxy-4-(4-pyridyl-sulfonyl)phenyl]-2-methyl-3,3,3-trifluoropropanamide, 2-hydroxy-2-methyl-N-[2-nitro-4-(phenyl-sulfonyl)phenyl]-3,3,3-trifluoropropanamide, S-(−)-N-(4-benzoyl-2-methylphenyl)-2-hydroxy-2-methyl-3,3,3-trifluoropropanamide, N-(4-benzoyl-2-chlorophenyl)-2-hydroxy-2-methyl-3,3,3-trifluoropropanamide, N-(4-benzoyl-2-bromophenyl)-2-hydroxy-2-methyl-3,3,3-trifluoropropanamide, N-(4-benzoyl-2-cyanophenyl)-2-hydroxy-2-methyl-3,3,3-trifluoropropanamide, N-(4-benzoyl-2-methoxyphenyl)-2-hydroxy-2-methyl-3,3,3-trifluoropropanamide, N-(4-benzoyl-2-hydroxyphenyl)-2-hydroxy-2-methyl-3,3,3-trifluoropropanamide, 2-hydroxy-N-[2-hydroxy-4-(4-pyridylsulfonyl)phenyl]-2-methyl-3,3,3-trifluoropropanamide, N-(4-benzoyl-2-fluorophenyl)-2-hydroxy-2-methyl-3,3,3-trifluoropropanamide, 2-hydroxy-N-[2-methoxy-4-(4-pyridyl-sulfonyl)phenyl]-2-methyl-3,3,3-trifluoropropanamide, 2-hydroxy-2-methyl-N-[2-nitro-4-(phenyl-sulfonyl)phenyl]-3,3,3-trifluoropropanamide, N-(4-benzoyl-2-chlorophenyl)-2-hydroxy-2-methyl-3,3,3-trifluoropropanamide, N-(4-benzoyl-2-bromophenyl)-3,3-difluoro-2-(difluoromethyl)-2-hydroxypropanamide, -(4-benzoyl-2-bromophenyl)-2-hydroxy-2-methyl-3,3,3-trifluoropropanamide, N-(4-benzoyl-2-methoxyphenyl)-2-hydroxy-2-methyl-3,3,3-trifluoropropanamide, N-benzoyl-2-hydroxyphenyl)-2-hydroxy-2-methyl-3,3,3-trifluoropropanamide and 2-hydroxy-N-[2-hydroxy-4-(4-pyridylsulfonyl)phenyl]-2-methyl-3,3,3-trifluoropropanamide,
and pharmaceutically acceptable in vivo cleavable esters of said compounds,
and pharmaceutically acceptable salts of said compounds and said esters.

24. The composition of claim 21 wherein the PDH elevator elevates human PDH activity.

25. A method of causing adipocytes to enhance production of adiponectin and leptin, comprising:
contacting adipocytes with both a pyruviate dehydrogenase kinase (PDHK) inhibitor and a PDH activity elevator for a period of time and under conditions such that PDHK is inhibited and PDH activity is elevated and production of adiponectin and leptin is enhanced.

26. The method of claim 25, wherein the adipocyte is maintained at a temperature in a range of from about 35° C. to about 40° C. at atmospheric pressure 110%.

27. The method of claim 25, wherein the adipocytes are present in a human.

28. The method of claim 25, further comprising:
transfecting the adipocyte with an antisense sequence which reduces PDHK production.

29. The method of claim 25, further comprising:
transfecting the adipocytes with a sequence encoding malic enzyme.
